(19) United States
(12) Patent Application Publication
(10) Pub. No.: US 2015/0018380 A1
(43) Pub. Date: Jan. 15, 2015

(54) Methods of Screening for Activation Deaminase Inhibitors Through Nuclear Import Inhibitors

(71) Applicant: Harold C. Smith, Rochester, NY (US)

(72) Inventor: Harold C. Smith, Rochester, NY (US)

(73) Assignee: University of Rochester, Rochester, NY (US)

(21) Appl. No.: 14/375,569

(22) PCT Filed: Feb. 7, 2013
(86) PCT No.: PCT/US13/25088

§ 371 Date: Jul. 30, 2014

Related U.S. Application Data
(60) Provisional application No. 61/596,030, filed on Feb. 7, 2012.

(51) Int. Cl. G01N 33/50

(52) U.S. Cl. CPC G01N 33/5035; G01N 2500/10; G01N 2500/04
USPC 514/285; 506/10; 558/397; 514/520; 548/468; 514/414; 546/70; 564/362; 514/649; 548/455

(57) ABSTRACT

The present invention provides methods for obtaining specific and non-toxic inhibitors of AID nuclear import. The methods comprise a primary screen and a counter screen to identify a pool of AID specific nuclear import inhibitors that do not have off-target of toxic effects. AID specific nuclear import inhibitors identified by the screens of the invention prevent nuclear entry, limit the access of AID to genomic DNA, and inhibit AID mutagenic activity. Preparations, including pharmaceutical preparations, comprising specific nuclear import inhibitors, used for example, to inhibit cancer progression, are also encompassed in the invention.

### CROSS-REFERENCE TO RELATED APPLICATION

This application claims priority to U.S. Provisional Application Ser. No. 61/596,030, filed Feb. 7, 2012, the contents of which are incorporated by reference herein in their entirety.

### BACKGROUND OF THE INVENTION

Antibody diversity in mammals is generated in two distinct phases. Developing B lymphocytes in the bone marrow assemble antibody variable regions via juxtaposing of component gene segments through the V(D)J DNA recombination process, giving rise to the primary antibody repertoire (Alt et al., 1992, Immunology Today, 13: 306-314; Willerford et al., 1996, Curr Opin Genet Dev, 6(5): 603-9). Later, activated peripheral B cells involved in immune responses can undergo two further mechanisms: somatic hypermutation (SHM) that introduces high rates of nucleotide substitutions in the variable region exons, and class switch recombination (CSR), which replaces the IgM-encoding Cμ segment with one of the genes for IgG, IgE and IgA antibodies (Honjo et al., 2002, Annu Rev Immunol, 20: 165-96.). Activation Induced cytidine Deaminase (AID) knockout in mice (Muramatsu et al., 1999, Journal of Biological Chemistry, 1999. 274: 18470-18476.) or targeted AID gene mutation (based on the location of mutations found in hyper-IgM syndrome patients deficient in SHM/CSR) abolished both CSR and SHM (Muramatsu et al., 2000, Cell, 102: 553-563; Revy et al., 2000, Cell 102: 565-75.). High levels of AID expression are restricted in vivo to activated B cells in germinal centers (Muramatsu et al., 1999, Journal of Biological Chemistry, 1999. 274: 18470-18476). Transcription factors such as NFκB, Stat6, E2A proteins and their negative regulators Id2 and Id3, and Pax5 that are linked to cell activation and cytokine signaling regulate transcription of AID in germinal centers (Sayegh et al., 2003, Nature Immunology, 4(6): 586-93; Dedeoglu et al., 2004, Int Immunol, 16(3): 395-404; Gonda et al., 2003, J Exp Med, 2003. 198(9): 1427-37; Endo et al., 2007, Oncogene, 26(38): 5587-95; Perez-Duran et al., 2007, Carcinogenesis, 2007. 28(12): 2427-33). Ectopic expression of AID induced SHM and CSR on reporter constructs even in non-lymphoid cells (Okazaki et al., 2002, Nature, 416: 340-5; Yoshikawa et al., 2002 Science, 296: 2033-6).

Sequence analysis showed that AID belongs to a growing family of cytidine deaminases whose prototypical member, APOBEC1, acts as an RNA editing enzyme on the apolipoprotein B mRNA in the nucleus (Wedekind et al., 2003, Trends in Genetics, 19: 207-16). AID has a single zinc-dependent catalytic domain that is necessary for ssDNA binding and dC to dU deamination (a characteristic of the entire APOBEC family) (Wedekind et al., 2003, Trends in Genetics, 19: 207-16). Although functionally distinct, SHM and CSR are mechanistically related by their dependence on AID-catalyzed dC to dU deamination. SHM and CSR also required uracil-DNA glycosylase (UNG) implicating the repair of dU in these processes (Harris et al., 2002, Molecular Cell, 10: 1247-53; Di Noia et al., 2002, Nature, 419: 43-8; Rada et al., 2002, Current Biology, 12: 1748-55; Petersen-Mahrt et al., 2002, Nature, 418: 99-103; Bransteitter et al., 2003, Proc Natl Acad Sci USA, 100: 4102-4107; Ramiro et al., 2003, Nature Immunology, 4: 452-6; Nambu et al., 2003, Science, 302(5653): 2137-40; Chaudhuri et al., 2003, Nature, 422: 726-30; Pham et al., 2003, Nature, 2003. 424(6944): 103-7).

AID shuttles between the cytoplasm and the nucleus (Rada et al., 2002, Proc Natl Acad Sci USA, 99(10): 7003-8; Ito et al., 2004, Proc Natl Acad Sci USA, 101(7): 1975-80; McBride et al., 2004, J Exp Med, 199(9): 1235-44; Brar et al., 2004, J Biol Chem, 279(25): 26395-26401; Patenaude et al., 2009, Nat Struct Mol Biol, 16(5): 517-27) and its ability to enter the nucleus is under stringent control, such that the vast majority of the protein is localized in the cytoplasm (Patenaude et al., 2009, Nat Struct Mol Biol, 16(5): 517-27). Shuttling of AID is considered to be mechanistically important for site-specific AID-dependent mutations, CSR and SHM (Ito et al., 2004, Proc Natl Acad Sci USA, 101(7): 1975-80; McBride et al., 2004, J Exp Med, 199(9): 1235-44; Patenaude et al., 2009, Nat Struct Mol Biol, 16(5): 517-27; Shinkura et al., 2004, Nature Immunology, 5(7): 707-12; Orthwein et al., 2010, J Exp Med, 207(12): 2751-65; Barreto et al., 2003, Mol Cell, 2003. 12(2): 501-8; Ta et al., 2003, Nature Immunology, 4(9): 843-8; Basu et al., 2009, Biochem Soc Trans, 37(Pt 3): 561-8; McBride et al., 2008, J Exp Med, 205(11): 2585-94). AID does not diffuse into the nucleus but rather uses energy-dependent, facilitated nuclear import and export chaperones (Orthwein et al., 2010, J Exp Med, 207(12): 2751-65; Chaudhuri et al., 2004, Nature, 430: 992-8). Nuclear import of nondiffusible macromolecules such as AID is mediated by a family of proteins (nucleoporins) comprising the nuclear pore complex (Walde et al., 2010, Trends Cell Biol, 20(8): 461-9) and a family of cargo receptor proteins known as karyopherins, transportins or importins. These chaperones form a variety of α/β heterodimers that bind cargoproteins (α subunit) via the cargo protein NLS and dock them (β subunit) to the nucleoporins. Although the nucleoporins and karyopherins mediate nuclear import of many diverse proteins and protein complexes (Kutay et al., 1997, EMBO J, 16(6): 1153-63), they have the ability to differentiate among proteins to selectively regulate entry into the nucleus (Walde et al., 2010, Trends Cell Biol, 20(8): 461-9; Debler et al., 2009, Nat Struct Mol Biol, 16(5): 457-9; Shah et al, 1998, Curr Biol, 8(25): 1376-86).

AID does not have a consensus nuclear localization sequence (NLS) composed of the canonical bipartite organization of basic amino acids but instead, basic residues are dispersed in four clusters (Patenaude et al., 2009, Nat Struct Mol Biol, 16(5): 517-27). The N-terminus of AID alone was not sufficient to localize chimeric reporter proteins to nucleus (Patenaude et al., 2009, Nat Struct Mol Biol, 16(5): 517-27). Nuclear import of AID depended on multiple and nonconsecutive charged protein motifs within the N-terminus and central region of AID that were not homologous to other APOBEC proteins (Patenaude et al., 2009, Nat Struct Mol Biol, 16(5): 517-27). It has been suggested that AID has a unique “conformational NLS” composed of interactions of the individual charged regions to form a super-secondary structure. Co-immunoprecipitation analysis showed that the region comprising the N-terminal to central portion of AID was required for binding to importins α1, α3, and α5 (Patenaude et al., 2009, Nat Struct Mol Biol, 16(5): 517-27). Point or deletion mutations within this region inhibited the interaction. PKA-dependent phosphorylation of two serines in AID's N-terminus plays a role in AID shuttling. Deaminase activity in the cell nucleus was enhanced by PKA-mediated phosphorylation (Basu et al., 2005, Nature, 438(7067): 508-11; Pasqualucci et al., 2006, Proc Natl Acad Sci USA, 103(2): 395-400) as was the interaction with DNA-targeting co-factors, such as replication protein A, RPA (Chaudhuri et al., 2004, Nature, 430: 992-8). Mutation of AID phosphorylation sites reduced nuclear import (Basu et al., 2009, Biochem Soc Trans, 37(Pt 3): 561-8; McBride et al., 2008, J Exp Med, 205(11): 2585-94; Chaudhuri et al., 2004, Nature, 430: 992-8; Cheng et al., 2009, Proc Natl Acad Sci USA, 106(8): 2717-22; Gazumyan et al., 2011, Mol Cell Biol, 31(3): 442-9) and inhibited CSR and SHM (Shinkura et al., 2004, Nature Immunology, 5(7): 707-12; Cheng et al., 2009, Proc Natl Acad Sci USA, 106(8): 2717-22; Gazumyan et al., 2011, Mol Cell Biol, 31(3): 442-9).

Cargo proteins are dissociated from the karyopherins in the nucleus by the GTP-bound form of Ran GTPase which shuttles with exported proteins back to the cytoplasm in the GDP-bound state (Debler et al., 2009, Nat Struct Mol Biol, 16(5): 457-9; Cook et al., 2010, Curr Opin Struct Biol, 20(2): 247-52; Yoneda et al., 1999, Cell Struct Funct, 24(6): 425-33). The gradient of high GTP-Ran in the nucleus to high GDP-Ran in the cytoplasm is mechanistically important for AID shuttling. AID is actively transported out of the nucleus through the interaction between a leucine-rich nuclear export signal motif within AID's C-terminus which binds to the export chaperone CRM1 (Rada et al., 2002, Proc Natl Acad Sci USA, 99(10): 7003-8; Ito et al., 2004, Proc Natl Acad Sci USA, 101(7): 1975-80; McBride et al., 2004, J Exp Med, 199(9): 1235-44; Barreto et al., 2003, Mol Cell, 2003. 12(2): 501-8; Ta et al., 2003, Nature Immunology, 4(9): 843-8). This process is inhibited by Leptomycin B (LMB) (Patenaude et al., 2009, Nat Struct Mol Biol, 16(5): 517-27; Bennett et al., 2006, Biochem Biophys Res Commun, 350(1): 214-9; Bennett et al., 2008, Journal of Biological Chemistry, 283(12): 7320-7; Ichikawa et al., 2006, J Immunol, 177(1): 355-61). Once in the cytoplasm, AID binds to HSP90 and this interaction actively retains AID in the cytoplasm (Orthwein et al., 2010, J Exp Med, 207(12): 2751-65) while protecting it from degradation (Orthwein et al., 2010, J Exp Med, 207(12): 2751-65; Geisberger et al., 2009, Proc Natl Acad Sci USA, 106(16): 6736-41; Ellyard et al., 2011, European Journal of Immunology, 41(2): 485-90). The half-life of nuclear AID is significantly shorter than cytoplasmic AID due to ubiquitinylation-dependent degradation of AID in the nucleus and this serves as an additional control on AID mutagenic activity (Orthwein et al., 2010, J Exp Med, 207(12): 2751-65; Aoufouchi et al., 2008, J Exp Med, 205(6): 1357-68). In addition, AID mRNA stability is decreased by a microRNA-mediated mechanism (Dorsett et al., 2008, Immunity, 2008. 28(5): 630-8; Teng et al., 2008, Immunity, 2008. 28(5): 621-9). It is not known whether more than one of these regulatory mechanisms needs to fail for AID to induce the genetic instability associated with various cancers. Recent data show that the most highly mutated pol II transcripts in the genome that are also among those most commonly deleted or translocated in B cell cancers bear the hallmark of nearest neighbor nucleotide preferences or “hotspots” for AID mutation (Klein et al., 2011, Cell, 147(1): 95-106; Robbiani et al., 2008, Cell, 135: 1028-38; Chiarle et al., 2011, Cell, 147: 107-19). It is proposed that limiting AID nuclear import and thereby access to genomic DNA will reduce AID-dependent mutations, however there are no currently known tools to limit AID nuclear import.

There is thus a need in the art for a cell-based high content screening (HCS) assay system to screen and identify compounds that serve as AID-selective nuclear import inhibitors. The present invention addresses this unmet need in the art.

### SUMMARY OF THE INVENTION

In one embodiment, the present invention provides a method of screening a library of compounds to provide a pool of specific and non-toxic nuclear inhibitors for AID. The method comprises conducting a primary screen to evaluate an on-target effect of at least one compound from the library for the ability to inhibit nuclear import of AID, where the primary screen comprises administering the at least one compound from the library and a nuclear export inhibitor to a cell modified to comprise AID tagged with a detectable label. The method further comprises conducting a counter screen to evaluate an off-target effect of the at least one compound from the library for the ability to alter the cellular localization of a non-AID protein, where the counter screen comprises administering the at least one compound from the library and the nuclear export inhibitor to a cell modified to comprise the non-AID protein tagged with a detectable label. The non-AID protein is selected from the group consisting of histone H1 (H1), APOBEC1 Complementation Factor (ACF), heterogeneous nuclear ribonucleoprotein A1 (hnRNP A1), and transaldoase 1 (TALDO-1). The method further comprises selecting a compound from the library that exhibits the ability to inhibit nuclear import of AID and does not substantially alter the cellular localization of the protein that is not AID, thereby providing a pool of specific and non-toxic nuclear import inhibitors for AID. In one embodiment, the cell of the primary screen and the cell of the counter screen are the same cell, thereby allowing for a simultaneous primary screen and secondary screen. In one embodiment, the nuclear export inhibitor is selected from the group consisting of LMB and Ratjadone A.

In one embodiment, evaluating an on-target effect in the primary screen comprises detecting the cellular localization of AID and comparing 1) the cellular localization of AID detected in a control cell after administration of the nuclear export inhibitor and 2) the cellular localization AID detected in the cell of the primary screen after the administration of both the nuclear export inhibitor and the compound. The compound is identified as exhibiting activity of inhibiting nuclear import of AID when the nuclear to cytoplasmic ratio (N/C) of AID in the control cell after administration of the nuclear export inhibitor is substantially greater than the N/C of AID in the cell of the primary screen after administration of the nuclear export inhibitor and the compound.

In one embodiment, evaluating an off-target effect in the counter screen comprises detecting the cellular localization of the non-AID protein and comparing 1) the cellular localization of the non-AID protein in a control cell detected after administration of the nuclear export inhibitor and 2) the cellular localization of the non-AID protein in the cell of the counter screen detected after the administration of both the nuclear export inhibitor and the compound. The compound is identified as exhibiting activity of altering the cellular localization of the non-AID protein when the nuclear to cytoplasmic ratio (N/C) of the non-AID protein in the control cell after administration of the nuclear export inhibitor is substantially greater than the N/C of the non-AID protein in the cell of the counter screen after administration of the nuclear export inhibitor and the compound.

In one embodiment, the method further comprises conducting a counter screen to evaluate the toxicity of the at least one compound. In another embodiment, the method further comprises evaluating the ability for the at least one compound to inhibit somatic hypermutation (SHM). In another embodiment, the method further comprises evaluating the ability for the at least one compound to inhibit class switch recombination (CSR).

The present invention also provides a compound selected from a pool of specific and non-toxic nuclear import inhibitors for AID, where the pool is identified by the screening methods of the invention. In one embodiment, the compound prevents somatic hypermutation and class switch recombination. In another embodiment, the compound exhibits an anti-cancer property.

The present invention also provides a method of treating a subject diagnosed with cancer or a subject at risk for developing cancer, comprising administering to the subject an effective amount of a compound that is an AID-selective inhibitor of nuclear import. In one embodiment, the cancer is selected from the group consisting of Follicular Lymphoma, chronic lymphocytic leukemias, acute lymphoblastic leukemias, diffuse large B cell lymphomas, Burkiett's mantle cell lymphomas, Hodgkin's disease, and any combination thereof. In another embodiment, the cancer is associated with a solid tumor of at least one member of the group consisting of the gastrointestinal system, urogenital system, breast, skin, and nervous system. In one embodiment, the method inhibits the progression of cancer.

The invention also provides a method of screening a library to provide a pool of specific and non-toxic nuclear import inhibitors for a first protein, where the first protein is capable of nuclear import. The method comprises conducting a primary screen to evaluate an on-target effect of at least one compound from the library for the ability to inhibit nuclear import of the first protein, where the primary screen comprises administering the at least one compound from the library and a nuclear export inhibitor to a cell comprising the first protein tagged with a detectable label. The method further comprises conducting a counter screen to evaluate an off-target effect of the at least one compound from the library for the ability to altering the cellular localization of a second protein, where the counter screen comprises administering the at least one compound from the library and a nuclear export inhibitor to a cell comprising a second protein tagged with a detectable label. The method further comprises selecting a compound from the library that exhibits the ability to inhibit nuclear import of the first protein and does not substantially altering the cellular localization of the second protein, thereby providing a pool of specific and non-toxic nuclear import inhibitors for the first protein. In one embodiment, the first protein is AID. In another embodiment, the cell of the primary screen and the cell of the counter screen are the same cell, thereby allowing for a simultaneous primary screen and secondary screen. In another embodiment, the nuclear export inhibitor is selected from the group consisting of LMB and Ratjadone A.

In one embodiment, evaluating an on-target effect in the primary screen comprises detecting the cellular localization of the first protein and comparing 1) the cellular localization of the first protein in a control cell detected after administration of the nuclear export inhibitor and 2) the cellular localization of the first protein in the cell of the primary screen detected after the administration of both the nuclear export inhibitor and the compound. The compound is identified as exhibiting activity of inhibiting nuclear import of the first protein when the nuclear to cytoplasmic ratio (N/C) of the first protein in the control cell after administration of the nuclear export inhibitor is substantially greater than the N/C of the first protein in the cell of the primary screen after administration of the nuclear export inhibitor and the compound.

In one embodiment, evaluating an off-target effect in the counter screen comprises detecting the cellular localization of second protein and comparing 1) the cellular localization of the second protein in a control cell detected after administration of the nuclear export inhibitor and 2) the cellular localization of the second protein in the cell of the counter screen detected after the administration of both the nuclear export inhibitor and the compound. The compound is identified as exhibiting activity of altering the cellular localization of the second protein when the nuclear to cytoplasmic ratio (N/C) of the second protein in the control cell after administration of the nuclear export inhibitor is substantially greater than the N/C of the second protein in the cell of the second protein after administration of the nuclear export inhibitor and the compound.

In one embodiment, the second protein is selected from the group consisting of histone H1 (H1), APOBEC1 Complementation Factor (ACF), heterogeneous nuclear ribonucleoprotein A1 (hnRNP A1), and transaldoase 1 (TALDO-1). In another embodiment, the primary screen and counter screen are conducted sequentially. In one embodiment, the primary screen narrows the library to provide a second library comprising essentially of at least one compound that exhibits the activity of inhibiting nuclear import of the first protein, and wherein the counter screen evaluates off target effects of the at least one compound of the second library.

In one embodiment, the method comprises conducting a counter screen to evaluate the toxicity of the at least one compound. In another embodiment, the method comprises evaluating the ability for the at least one compound to inhibit somatic hypermutation (SHM). In another embodiment, the method further comprises evaluating the ability for the at least one compound to inhibit class switch recombination (CSR).

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a schematic that illustrates the principle behind the EGFP-AID Nuclear import HCS assay. The assay is based on the simultaneous primary screen for AID nuclear import and a counter screen in the same cell for nuclear import of a different protein that is considered off-target.

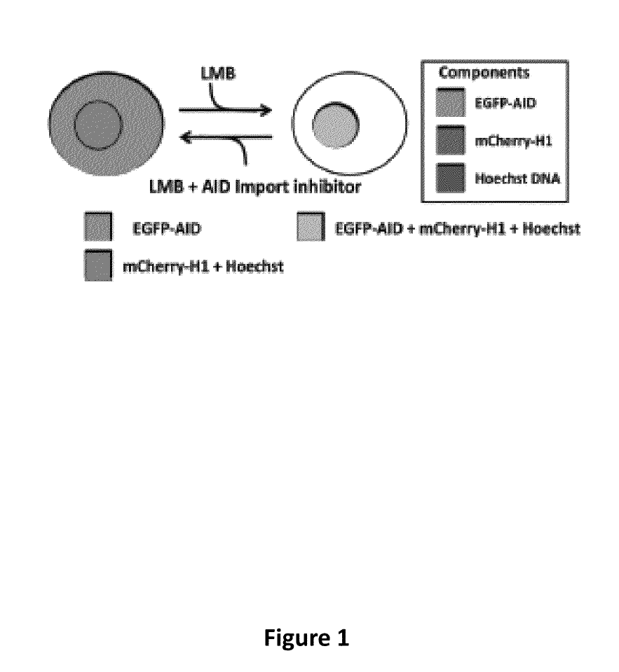

FIG. 2, comprising FIG. 2A through FIG. 2F, depicts images from an AID-EGFP high content screening assay. FIG. 2A and FIG. 2B depict Hoechst staining (FIG. 2A) and predominant cytoplasmic AID-EGFP localization (FIG. 2B) in untreated cells. FIG. 2C and FIG. 2D depict Hoechst staining (FIG. 2C) and nuclear AID-EGFP localization (FIG. 2D) in cells treated with 100 ng/mL LMB for 180 minutes. FIG. 2E and FIG. 2F depict Hoechst staining (FIG. 2E) and nuclear AID-EGFP localization (FIG. 2F) in cells treated with 100 ng/mL Ratjadone A for 180 minutes.

FIG. 3 depicts the image segmentation into masks using the Molecular Translocation and Translocation Enhanced image analysis used to determine the nuclear and cytoplasmic distributions of Hoechst, EGFP-AID, and mCherry-H1 channels as an example of how changes in nuclear/cytoplasmic (N/C) ratio of protein distributions are quantified.

FIG. 4, comprising FIG. 4A and FIG. 4B, illustrates the expression of transfected proteins in cells of the primary and/or counter screens. FIG. 4A depicts the results of FACS analysis showing that cells co-expressed both EGFP-AID and mCherry-H1 proteins. FIG. 4B shows western blots illustrating the presence of AID and H1 proteins (top panels) or ACF and A1 proteins (bottom panels) in HEK293T cells of the HCS assay.

FIG. 5, comprising FIG. 5A through FIG. 5U, illustrates the presence of transfected proteins in cells of the primary and/or counter screens of the assay, as detected by fluorescence microscopy. FIG. 5A through FIG. 5C show localization of EGFP, and mCherry signals as well as the merge of EGFP and mCherrry signal in cells expressing AID-EGFP and mCherry-H1 without LMB addition. FIG. 5D through FIG. 5F shows the selective redistribution of AID-EGFP in the cells expressing AID-EGFP and mCherry-H1 after LMB addition. FIG. 5G through FIG. 5I illustrates the distribution of EGFP and mCherry, as well as the merge of EGFP and mCherry in cells expressing ACF-EGFP and mCherry-hnRNP A1 without LMB addition. FIG. 5J through FIG. 5L shows the redistribution of ACF-EGFP in cells expressing ACF-EGFP and mCherry-hnRNP A1 after LMB addition. FIG. 5M through FIG. 5O shows the selective redistribution of mCherry-hnRNP A1 in cells expressing ACF-EGFP and mCherry-hnRNP A1 after osmotic stress of the cultures. FIG. 5P through FIG. 5R show localization of EGFP, and mCherry signals as well as the merge of EGFP and mCherrry signal in cells expressing AID-EGFP and mCherry-TALDO1 without LMB addition. FIG. 5S through FIG. 5U shows the selective redistribution of AID-EGFP in the cells expressing AID-EGFP and mCherry-TALDO1 after LMB addition.

FIG. 6 is a scatter plot used to validate the assays ability to identify with discrimination, compounds affecting AID nuclear import based on a screen of the 1,280 compound LOPAC library in 4×384 well plates. Five compounds, including SID 53777289, were identified as compounds which produced greater than 50% inhibition of AID nuclear import.

FIG. 7, comprising FIG. 7A through FIG. 7C, depicts the results of B cell CSR and SHM functional endpoint analyses. FIG. 7A depicts a set of graphs illustrating IgM to IgA switching in stimulated CH12F3 cells, as measured by flow cytometry. FIG. 7B is a graph depicting IgG1 switching in mouse primary B cells, four days after stimulation. FIG. 7C is a set of graphs illustrating the expression of GFP and AID in and AID−/− subline of DT40 that have been complemented with human AID-IRES-GFP.

FIG. 8 is a set of images depicting Hoechst staining and AID-EGFP localization, along with a color-combined image, in untreated and LMB (200 ng/mL) treated AID-EGFP expressing cells.

FIG. 9 depicts a heat map of 4×384-well plates from a first run of a screen of compounds from the LOPAC library.

FIG. 10 depicts a scatter plot of the 4×384-well plates illustrating the percent AID import inhibition from a run of a screen of compounds from the LOPAC library.

FIG. 11 depicts an overlay scatter plot of all 4 plates used in the screen of compounds from the LOPAC library, illustrating the identification of five compounds which induced greater than 50% inhibition of AID nuclear import.

FIG. 12 is a scatter plot depicting the identification of compounds that were cytotoxic outliers in the screen of compounds from the LOPAC library.

FIG. 13 is a scatter plot depicting the identification of compounds that are Hoechst fluorescent outliers in the screen of compounds from the LOPAC library.

FIG. 14 is a scatter plot depicting the identification of compounds that are EGFP nuclear fluorescent outliers in the screen of compounds from the LOPAC library.

FIG. 15 is a scatter plot depicting the identification of compounds that are EGFP cytoplasmic fluorescent outliers in the screen of compounds from the LOPAC library.

FIG. 16 depicts performance statistics of two different runs of the screen of compounds of the LOPAC library demonstrating that the consistency of the screen.

FIG. 17 is a graph depicting the percent inhibition of AID nuclear import mediated by the LOPAC compounds in two different runs of the screen. As depicted, data from the runs produced an R2 value of 0.90 demonstrating the consistency and reproducibility of the screen.

FIG. 18 depicts the structure of Bay 11-7085 (Pubchem SID 53777289) as well as images of Hoechst staining, AID-EGFP localization, and color combine, of cells treated with Bay 11-7085.

FIG. 19 depicts the structure of SU 5416 (Pubchem SID 53778216) as well as images of Hoechst staining, AID-EGFP localization, and color combine, of cells treated with SU 5416.

FIG. 20 depicts the structure of Ellipticine (Pubchem SID 53777637) as well as images of Hoechst staining, AID-EGFP localization, and color combine, of cells treated with Ellipticine.

FIG. 21 depicts the structure of Mitoxantrone (Pubchem SID 53777885) as well as images of Hoechst staining, AID-EGFP localization, and color combine, of cells treated with Mitoxantrone.

FIG. 22 depicts the structure of SU 6656 (Pubchem SID 53778085) as well as images of Hoechst staining, AID-EGFP localization, and color combine, of cells treated with SU 6656.

FIG. 23, comprising FIG. 23A and FIG. 23B, illustrates the expression of proteins in cells of the primary and/or counter screens. FIG. 23A, depicts the results of FACS analysis showing that cells co-expressed both AID-EGFP and mCherry-TALDO1 proteins. FIG. 23B shows western blots illustrating the presence of AID-EGFP and mCherry-TALDO1 in the HeLa cells of the assay. The cells are used in a simultaneous primary and counter screen to identify AID specific nuclear import inhibitors.

FIG. 24, comprising FIG. 24A through FIG. 24F, is a set of images depicting the localization of AID and TALDO1 in HeLa cells modified to stably express both AID-EGFP and mCherry-TALDO1. FIG. 24A through FIG. 24C depicts localization in control conditions, while FIG. 24D through FIG. 24F depicts localization after treatment with LMB. The cells are used in a simultaneous primary and counter screen to identify AID specific nuclear import inhibitors.

### DETAILED DESCRIPTION

The present invention relates to systems and methods to screen for an inhibitor of the nuclear import of Activation Induced cytidine Deaminase (AID). In one embodiment, the inhibitor of the invention is a novel regulator of immunoglobulin diversification. In another embodiment, the inhibitor of the invention is an anti-cancer compound. For example, the inhibitor identified by the screen of the invention can be used to prevent, delay, or inhibit cancer progression. In some instances, the novel regulator can be used as a scaffold to generate additional novel compounds.

AID mutates genomic DNA, which has an important physiological role in the generation of antibody diversity. However, aberrant AID activity is also linked to unregulated mutagenesis leading to cancer. The present invention is related to the generation of high content screening methods to identify specific and non-toxic inhibitors of AID nuclear import that do not affect the nuclear import of other proteins. AID specific nuclear import inhibitors, as identified by the methods of the present invention, limit the entry of AID to the nucleus while permitting nuclear export of AID, thereby limiting the amount of AID that has access to genomic DNA. Restricting AID nuclear entry inhibits and/or limits unregulated AID mediated mutagenesis associated with multiple forms of cancer.

The methods and assays of the invention comprise automated high content screening methods, using a live cell assay to screen large quantities of test compounds for their ability to influence cellular localization of AID. In one embodiment, the methods of the invention comprise a primary screen that identifies inhibitors of AID nuclear import. In one embodiment, the methods of the invention further comprise one or more counter screens that are used to evaluate the specificity and toxicity of potential AID nuclear import inhibitors identified from the primary screen. In one embodiment, the one or more counter screens are built-in to the cells of the primary screen, wherein a test compound is evaluated for its ability to influence cellular localization of AID while not affecting the localization of a non-AID element. Thus, in one embodiment, the invention comprises a live cell assay that simultaneously expresses AID and the non-AID element, allowing for simultaneous primary and counter screens. In another embodiment, the one or more counter screens are performed on a separate cell population, subsequent to performing the primary screen. The primary and counter screens of the invention ensure the selection of compounds that bind to AID and inhibits in nuclear import rather than binding to the transport machinery itself.

The combination of the primary screen and the one or more counter screens allows for the identification of compounds that are AID-specific nuclear import inhibitors, thereby minimizing off target consequences associated with broad spectrum or non-selective nuclear import inhibitors.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “agent” includes any substance, molecule, element, compound, entity, or a combination thereof. It includes, but is not limited to, e.g., protein, oligopeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably. Further, a “test agent” or “candidate agent” is generally a subject agent for use in an assay of the invention.

The term “assessing” includes any form of measurement, and includes determining if an element is present or not. The terms “determining”, “measuring”, “evaluating”, “assessing” and “assaying” are used interchangeably and may include quantitative and/or qualitative determinations. Assessing may be relative or absolute. “Assessing binding” includes determining the amount of binding, and/or determining whether binding has occurred (i.e., whether binding is present or absent).

The term “binding” refers to a direct association between at least two molecules, due to, for example, covalent, electrostatic, hydrophobic, ionic and/or hydrogen-bond interactions under physiological conditions.

“Contacting” refers to a process in which two or more molecules or two or more components of the same molecule or different molecules are brought into physical proximity such that they are able undergo an interaction. Molecules or components thereof may be contacted by combining two or more different components containing molecules, for example by mixing two or more solution components, preparing a solution comprising two or more molecules such as target, candidate or competitive binding reference molecules, and/or combining two or more flowing components. Alternatively, molecules or components thereof may be contacted combining a fluid component with molecules immobilized on or in a substrate, such as a polymer bead, a membrane, a polymeric glass substrate or substrate surface derivatized to provide immobilization of target molecules, candidate molecules, competitive binding reference molecules or any combination of these. Molecules or components thereof may be contacted by selectively adjusting solution conditions such as, the composition of the solution, ion strength, pH or temperature. Molecules or components thereof may be contacted in a static vessel, such as a microwell of a microarray system, or a flow-through system, such as a microfluidic or nanofluidic system. Molecules or components thereof may be contacted in or on a variety of media, including liquids, solutions, colloids, suspensions, emulsions, gels, solids, membrane surfaces, glass surfaces, polymer surfaces, vesicle samples, bilayer samples, micelle samples and other types of cellular models or any combination of these.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “downstream” or “upstream” with respect to a signaling pathway is based on epistatic relationships in a linear signaling cascade: if “A” activates “B” and “B” activates “C”, the “A” is upstream of “B” and “B” is upstream of “C”. Similarly, “B” is downstream of “A” and “C” is downstream of “B”.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), enzymatic labels (e.g., horseradish peroxidase, beta-galactosidase, luciferase, alkaline phosphatase), biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

As used herein, an “inhibitory-effective amount” is an amount that results in a detectable (e.g., measurable) amount of inhibition of an activity of AID nuclear import. In some instance, the activity of AID nuclear import is its ability to bind with a corresponding chaperone. In other instances, the activity of AID refers to its ability to induce class switch recombination (CSR) or somatic hypermutation (SHM).

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

The term “interact” or “interaction” refers to a measurable chemical or physical interaction between a target molecule and a candidate molecule that is capable of affecting the structure and/or composition of a target molecule, a candidate molecule or both such that the biological activity of the target molecule, the candidate molecule or both is affected. Interactions capable of affecting the structure and/or composition of a molecule include, but are not limited to, reactions resulting in the formation of one or more covalent bonds, resulting in the breaking of one or more covalent bonds, electrostatic associations and repulsions, formation and/or disruption of hydrogen bonds, formation and/or disruption of electrostatic forces such as dipole-dipole interactions, formation and/or disruption of van der Waals interactions or processes comprising combinations of these.

“Molecule” refers to a collection of chemically bound atoms with a characteristic composition. As used herein, a molecule can be neutral or can be electrically charged. The term molecule includes biomolecules, which are molecules that are produced by an organism or are important to a living organism, including, but not limited to, proteins, peptides, lipids, DNA molecules, RNA molecules, oligonucleotides, carbohydrates, polysaccharides; glycoproteins, lipoproteins, sugars and derivatives, variants and complexes of these, including labeled analogs of these having one or more vibrational tag. The term molecule also includes candidate molecules, which comprise any molecule that it is useful, beneficial or desirable to probe its capable to interact with a molecule such as a target molecule. Candidate molecules include therapeutic candidate molecules which are molecules that may have some effect on a biological process or series of biological processes when administered. Therapeutic candidate molecules include, but are not limited to, drugs, pharmaceuticals, potential drug candidates and metabolites of drugs, biological therapeutics, potential biological therapeutic candidates and metabolites of biological therapeutics, organic, inorganic and/or hybrid organic-inorganic molecules that interact with one or more biomolecules, molecules that inhibit, decrease or increase the bioactivity of a biomolecule, inhibitors, ligands and derivatives, variants and complexes of these. The term molecule also includes target molecules, which comprise any molecule that it is useful, beneficial or desirable to probe its capable to interact with a molecule such as a candidate molecule. Target molecules useful for identifying, characterizing and/or optimizing therapeutics and therapeutic candidates comprise biomolecules, and derivatives, variants and complexes of biomolecules. The term molecule also includes competitive binding reference molecules. Competitive binding reference molecules useful in the present invention are molecules that are known to bind, at least to some extent, to a target molecule, and in some embodiments comprise a known drug, biological therapeutic, biomolecule, lead compound in a drug discovery program, and derivatives, variants, metabolites and complexes of these.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Screening” referred to in the present invention includes not only so-called first screening for selecting the inhibitor of the present invention among a plurality of candidates, but also a counter screen for selecting the inhibitor of the present invention among a plurality of candidates. In some instances, the target in the screen of the invention is AID. Accordingly, target effects refer to AID subcellular localization with or without LMB treatment. In some instances, the off target in the screen refers to any other protein that is either always localized in the nucleus (e.g., Histone) or shuttles between the nucleus and cytoplasm (e.g., ACF, A1 and TALDO1). Accordingly, off target effects refer to compounds that in addition to AID or solely affect the subcellular localization of any of the counter screen proteins.

“Primary screen” as referred to herein comprises assays for the acquisition of images of cells to detect AID localization. Localization of AID is made through the detection of a signal corresponding to AID. In some instances, the primary screen relates to a live cell assay that can be used to sample a chemical library, preferably in high throughput fashion for inhibitors of AID nuclear import, i.e. changes in AID N/C ratio in LMB treated cells.

“Counter screen” as referred to herein comprises an assay that can be used to evaluate a population of test compounds, identified by the primary screen as being inhibitors of nuclear import of AID, for being specific and selective inhibitors of AID nuclear import. In some instances, the counter screen relates to an assay that is used to evaluate hits from the primary for off target effects that may mistakenly cause a change in the AID N/C but are due to mechanism that effect cellular other cellular functions and only indirectly lead to changes in AID N/C ratio. For example, Histone, A1, ACF and TALDO1 are evaluated to rule out affects on other transporters and transported proteins. The primary screen is unique in that the histone counter screen is co-expressed. The A1/ACF counter screen seeks to rule out effects on transporters unrelated to those used for AID nuclear import. The AID/TALDO1 assays uses TALDO1 as a counter screen to rule out hits that inhibit the transporters that AID and TALDO 1 have in common and selects for hits that inhibit AID nuclear import because the bind to AID and thereby inhibit its interaction with nuclear import machinery.

“Secondary screen” as referred to herein comprises assays for evaluating compounds for their AID selective interactions by quantifying their ability of the hits from the primary screen to inhibit cellular processes that are downstream and dependent on AID nuclear localization. In some instances, the secondary screen relates to an orthogonal live cell assay that is used to assess hits from the primary screen for their ability to affect functional endpoints that are downstream and therefore dependent on AID nuclear import i.e. CSR or SHM.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species.

“Test agents” or otherwise “test compounds” as used herein refers to an agent or compound that is to be screened in one or more of the assays described herein. Test agents include compounds of a variety of general types including, but not limited to, small organic molecules, known pharmaceuticals, polypeptides; carbohydrates such as oligosaccharides and polysaccharides; polynucleotides; lipids or phospholipids; fatty acids; steroids; or amino acid analogs. Test agents can be obtained from libraries, such as natural product libraries and combinatorial libraries. In addition, methods of automating assays are known that permit screening of several thousands of compounds in a short period.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition associated with AID nuclear import, including alleviating symptoms of such diseases.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to methods of screening test compounds for specific inhibitors of AID nuclear import and uses of such inhibitors to prevent CSR-like and SHM-like mutations that lead to chromosomal mutations and translocations associated with the progression in cancer. In one embodiment, the invention comprises evaluating the change in the localization of elements of the screen in response to the addition of a test compound. Preferably, an element of the screen includes but is not limited to proteins, peptides, nucleic acids, and portions thereof, whose localization within a cell is evaluated in the screen of the invention. For example, in one embodiment, an element of the screen is AID. Preferably, the screen of the invention evaluates the activity of a test compound in the presence of a nuclear export inhibitor (e.g., LMB, Ratjadone A). In this aspect, high content screens of the invention comprise evaluating the change in the response of an element of the screen to the addition of a nuclear export inhibitor and prior addition of a test compound.

In one embodiment, the methods of the invention relate to high throughput screening methods and automated screening of large quantities of test compounds to identify specific inhibitors of AID nuclear import.

In one embodiment, the methods comprise a primary high content screen where the localization of AID protein within a live cell is determined by detecting a marker associated with AID localization.

In another embodiment, the methods of the present invention further comprise a counter screen to evaluate the specificity and toxicity of nuclear import inhibitors identified in the primary screen. In one embodiment, the counter screen comprises an additional high content screen wherein the localization of non-AID proteins is evaluated in response to a test compound. In this aspect, the counter screen identifies off target effects of test compounds or of nuclear import inhibitors identified in the primary screen. In one embodiment, the counter screen is coincident with the primary screen, where the cell expresses AID, whose localization is examined in the primary screen, and simultaneously expresses a non-AID protein, whose localization is examined in the counter screen. In one embodiment, expression of both AID and a non-AID protein in a single cell allows for the simultaneous determination of the “on” target effects on AID and “off” target effects on the non-AID protein of the screened test compounds. In another embodiment, the counter screen is performed on a separate cell population, different from those used in the primary screen, where test compounds identified in the primary screen are then tested for off-target effects on modulating localization of one or more non-AID proteins. In one embodiment, the non-AID protein of the counter screen is a primarily nuclear localized protein. In yet another embodiment, more than one counter screens are performed to better evaluate off-target effects and/or toxicity of test compounds.

The methods of the invention are related to the automated determination of the nuclear to cytoplasmic (N/C) ratio for the various elements of the screen before and after addition of a test compound. In one embodiment, N/C ratio of a given element is determined by evaluating the amount of the element in nuclear and cytoplasmic regions of a cell as seen in acquired images of the cell. In one embodiment, the given element is tagged with a marker, for example a fluorescent tag, such that localization of the element is observable using standard techniques known in the art.

In another aspect of the invention, the invention relates to selection of an AID specific nuclear import inhibitor from a library of test compounds, based on the ability for the test compound to retain cytoplasmic localization of AID when the cell is further treated with a nuclear export inhibitor (e.g. LMB, Ratjadone A). In one embodiment, the test compound that is identified as an inhibitor is selected following determining that the test compound does not alter the localization of non-AID proteins. In another embodiment, the test compound that is identified as an inhibitor is selected following determining that the inhibitor is non-toxic to a living cell.

The invention also provides an AID specific nuclear import inhibitor that can be used to treat or prevent forms of cancer, including but not limited to Follicular Lymphoma, chronic lymphocytic leukemias, acute lymphoblastic leukemias, diffuse large B cell lymphomas, Burkiett's mantle cell lymphomas, and Hodgkin's disease. For example, an AID nuclear import inhibitor identified by the screen of the invention can be used to prevent, delay, or inhibit cancer progression. An identified inhibitor may inhibit AID nuclear import through any known or unknown mechanism. For example, in one embodiment, an identified inhibitor antagonizes the interaction of AID with nuclear import machinery. In another embodiment, an identified inhibitor inhibits AID nuclear import through antagonism of the NFκB signaling pathway.

### AID

AID is a DNA mutator that is responsible for immunoglobulin (Ig) gene rearrangements known as class switch recombination (CSR) and hypermutation of the Ig variable region known as somatic hypermutation (SHM). These processes uniquely occur in germinal centers during B lymphocyte activation where de novo AID expression and AID shuttling from the cytoplasm to the cell nucleus are thought to be critical for regulated and targeted mutagenesis. In B cell and non B cell cancer cells AID frequently is constitutively expressed and expressed as protein variants that may have aberrant gene targeting and/or enhanced nuclear localization. AID mutagenic activity has been implicated as being responsible for mutations and recombinations characteristic of cancer cell genetic changes (McCord et al., 2011, Cell, 147(1): 20-2). The systems and methods of the present invention provide for a high content screening assay to identify compounds that selectively inhibit AID translocation into the nucleus. Such compounds identified by the present invention would be effective in reducing oncogenic mutations and chromosomal translocations. Further, identified compounds could be used as research probes to examine the role of AID in SHM, CSR, and oncogenic mutagenesis.

Recent data show that the most highly mutated pol II transcripts in the genome that are also among those most commonly deleted or translocated in B cell cancers bear the hallmark of nearest neighbor nucleotide preferences or “hotspots” for AID mutation (Klein et al., 2011, Cell, 147(1): 95-106). Non-limiting examples of candidate AID substrates now include C-MYC, PIM-1, BCl2, BCL6, β-catenin, PAX5, Socs1, Junb, RhoH/TTF, mir142 and the TP53 tumor suppressor gene (Klein et al., 2011, Cell, 147(1): 95-106; Pasqualucci et al., 2001, Nature, 412: 341-6; Pasqualucci et al., 2003, Blood, 101: 2914-23; Gaidano et al., 2003, Blood, 102: 1833-41; Okazaki et al., 2003, Journal of Experimental Medicine, 197: 1173-1181; Machida et al., 2004, Proc Natl Acad Sci USA, 101(12): 4262-7; Epeldegui et al., 2007, Mol Immunol, 44(5): 934-42; Matsumoto et al., 2007, Nat Med, 13(4): 470-6). The mutator phenotype and induction of neoplasias is a characteristic of most of the APOBEC family (Harris et al., 2002, Molecular Cell, 10: 1247-53) when these enzymes are overexpressed (Yamanaka et al., 1995, Proc. Natl. Acad. Sci USA, 92: 8483-8487; Yamanaka et al., 1997, Genes Dev, 11(3): 321-33; Bonvin et al., 2008, Curr Opin Infect Dis, 21(3): 298-303; Xu et al., 2007, Hepatology, 46(6): 1810-20). Constitutive AID expression has been shown in a number of B cell malignancies, including Follicular Lymphoma (FL) where a sustained expression of AID has been suggested to be causal in FL disease progression by inducing accumulation of oncogene mutations leading to chromosomal translocations (Hardianti et al., 2004, Leukemia, 18(4): 826-31; Smit et al., 2003, Cancer Res, 63(14): 3894-8). Elevated expression of AID has also been described in a subset of B cell chronic lymphocytic leukemias (CLL) and acute lymphoblastic leukemias (ALL), most diffuse large B cell lymphomas (DLBCL), some Burkitt's mantle cell lymphomas, as well as Hodgkin's disease (68. Hardianti et al., 2004, Leukemia, 18(4): 826-31; Smit et al., 2003, Cancer Res, 63(14): 3894-8; Greeve et al., 2003, Blood, 101: 3574-3580; McCarthy et al., 2003, Blood, 101: 4903-8; Oppezzo et al., 2003, Blood, 101: 4029-32; Babbage et al., 2004, Blood, 103(7): 2795-8; Lossos et al., 2004, Leukemia, 18: 1775-9; Pasqualucci et al., 2004, Blood, 104: 3318-25; Heintel et al., 2004, Leukemia, 18(4): 756-62; Feldhahn et al., 2007, J Exp Med, 204(5): 1157-66; Deutsch et al., 2007, Blood, 109(8): 3500-4; Lenz et al., 2007, J Exp Med, 204(3): 633-43; Hardianti et al., 2005, Eur J Haematol, 74(1): 11-9; Epeldegui et al., 2006, Curr Opin Oncol, 18(5): 444-8; Mottok et al., 2005, J Clin Pathol, 58(9): 1002-4; Greiner et al., 2005, J Pathol, 205(5): 541-7). AID expression has also been detected in non-lymphoid primary tumors, including breast, liver and gastric cancer (Babbage et al., 2006, Cancer Res, 66(8): 3996-4000; Kim et al., 2007, Tumour Biol, 28(6): 333-9; Kou et al., 2007, Int J Cancer, 120(3): 469-76; Komori et al., 2008, Hepatology, 47(3): 888-96), and a link between AID overexpression following chronic inflammation and tumorigenesis has been suggested (Perez-Duran et al., 2007, Carcinogenesis, 2007. 28(12): 2427-33; Matsumoto et al., 2007, Nat Med, 13(4): 470-6; Marusawa, 2008, Int J Biochem Cell Biol, 40(8): 1399-402).

Unlike other known mutator phenotypes that are due to defective repair of spontaneous DNA damage (Loeb, 2001, Cancer Res, 61(8): 3230-9; Loeb et al., 2003, Proc Natl Acad Sci USA, 100(3): 776-81) misregulated AID activity on genomic DNA directly causes genetic mutations (Klein et al., 2011, Cell, 147(1): 95-106; Robbiani et al., 2008, Cell, 135: 1028-38; Chiarle et al., 2011, Cell, 147: 107-19). However, the outcome in both cases is the accelerated accumulation of oncogenic mutations. In mice, ubiquitous transgenic expression of AID induces T cell lymphomas and lung adenocarcinomas (Okazaki et al., 2003, Journal of Experimental Medicine, 197: 1173-1181). Mouse AID was required for the generation of IL6- or pristane-induced plasmacytomas and for lymphomas in various transgenic strains (Ramiro et al., 2004, Cell, 118(4): 431-8; Unniraman et al., 2004, Nature Immunology, 5: 1117-23; Kotani et al., 2007, Proc Natl Acad Sci USA, 104(5): 1616-20; Pasqualucci et al., 2008, Nat Genet, 40(1): 108-12). In this model AID expression was haplo-insufficient and heterozygous mutants develop disease at a significantly lower rate than wild-type mice (Takizawa et al., 2008, J Exp Med, 205(9): 1949-57), implicating AID overexpression in the disease mechanism. In human CLL and in DLBCLs AID mRNA expression (McCarthy et al., 2003, Blood, 101: 4903-8; Oppezzo et al., 2003, Blood, 101: 4029-32) in the absence of ongoing Ig SHM (Greeve et al., 2003, Blood, 101: 3574-3580; Lossos et al., 2004, Leukemia, 18: 1775-9; Pasqualucci et al., 2004, Blood, 104: 3318-25; Damle et al., 1999, Blood, 94: 1840-7; Hamblin et al., 1999, Blood, 94: 1848-54; Lossos et al., 2000, Proc Natl Acad Sci USA, 97: 10209-13; Alizadeh et al., 2000, Nature, 403: 503-11; Rosenwald et al., 2002, New England Journal of Medicine, 346: 1937-47) was associated with a poor prognosis. Normal cellular mechanisms targeting AID activity to appropriate Ig genomic sites may have been altered. AID overexpression alone might have this effect because AID requires no cofactors to hypermutate DNA when overexpressed in E. coli (Petersen-Mahrt et al., 2002, Nature, 418: 99-103) or to scan and mutate ssDNA substrates in vitro (Pham et al., 2011, Journal of Biological Chemistry, 286(28): 24931-42).

Although ≧98% of AID mRNA is full length in germinal center B cells, alternatively spliced mRNAs were observed in circulating B cells, suggesting that alternative mRNA splicing may add an additional level of control over AID mutagenic activity. Alternatively spliced AID mRNA variants have been identified that do no express the exon encoding the deaminase domain and/or the exon encoding the nuclear export signal (Wu et al., 2008, Blood, 112(12): 4675-82). In circulating malignant B cells 28% of the total cellular AID mRNA was alternatively spliced to a form that lacked the exon encoding the nuclear export signal (Wu et al., 2008, Blood, 112(12): 4675-82; Iacobucci, et al., 2010, Leukemia, 24(1): 66-73). The C-terminal truncated AID protein expressed from this alternatively spliced mRNA was retained in the nucleus to a greater extent and supported SHM (consistent with SHM having the sole requirement of the deaminase domain) but no longer supported CSR (consistent with the requirement for protein-protein interactions within the C-terminus for CSR). Other reports claimed that although the C-terminal truncated form of AID had a greater nuclear distribution in cancer cells the deaminase activity was less active than that in wild type AID (van Maldegem et al., 2010, Journal of Immunology, 184(5): 2487-91; Marantidou et al., 2010, Blood cells, molecules & diseases, 44(4): 262-7).

AID access to the nucleus and thus to genomic DNA is highly regulated. Import of AID into the nucleus is mediated by nucleoporins that comprise the nuclear pore complex (Walde et al., 2010, Trends Cell Biol, 20(8): 461-9) and a family of cargo chaperone proteins known as karyopherins, transportins or importins. AID does not have a consensus nuclear localization sequence (NLS), but rather is thought to have a unique “conformational NLS” composed of interactions of individual charged regions of the AID polypeptide to form a secondary structure (Patenaude et al., 2009, Nat Struct Mol Biol, 16(5): 517-27). Once in the nucleus, AID activity is regulated by ubiquitinylation-dependent degradation, which limits the half-life of AID while in the nucleus (Orthwein et al., 2010, J Exp Med, 207(12): 2751-65; Aoufouchi et al., 2008, J Exp Med, 205(6): 1357-68). AID is actively transported out of the nucleus through interaction with an export chaperone, CRM1 (Rada et al., 2002, Proc Natl Acad Sci USA, 99(10): 7003-8; Ito et al., 2004, Proc Natl Acad Sci USA, 101(7): 1975-80; McBride et al., 2004, J Exp Med, 199(9): 1235-44; Barreto et al., 2003, Mol Cell, 2003. 12(2): 501-8; Ta et al., 2003, Nature Immunology, 4(9): 843-8), a process that is inhibited by Leptomycin B (LMB). Further AID regulation takes place in the cytoplasm, where AID binds to HSP90 which actively restricts AID to the nucleus (Orthwein et al., 2010, J Exp Med, 207(12): 2751-65) and prevents AID degradation (Orthwein et al., 2010, J Exp Med, 207(12): 2751-65; Geisberger et al., 2009, Proc Natl Acad Sci USA, 106(16): 6736-41; Ellyard et al., 2011, European Journal of Immunology, 41(2): 485-90). Despite these regulatory mechanisms, AID activity can, in some instances, become deregulated, leading to oncogenic mutagenesis. The present invention is based up on the development of a high content screening assay to identify compounds that inhibit nuclear import of AID, thereby restricting the access of AID to genomic DNA.

### High Content Screening (HCS) Assays

The present invention provides a system and method to screen and identify compounds that selectively inhibit nuclear import of AID. In one embodiment, the systems and methods comprise high content screening (HCS) of suitable compounds. In some instances, HCS is a screening method that uses live cells to perform a series of experiments as the basis for high throughput compound discovery. Typically, HCS is an automated system to enhance the throughput of the screening process. However, the present invention is not limited to the speed or automation of the screening process.

As described elsewhere herein, AID is a mutator of genomic DNA, whose activity, when deregulated, leads to oncogenic mutations. The present invention comprises an HCS assay to screen for compounds that inhibit AID mediated mutagenesis. In one embodiment, the compounds are screened for the ability to inhibit nuclear import of AID. Non-limiting examples of the mechanism of action for candidate compounds include prevention of the conformational NLS from forming, preventing PKA phosphorylation of AID, and preventing importin binding to AID.

In one embodiment, the HCS assay of the invention provides for a system to generate high quality “hits” identifying compounds that inhibit nuclear import of AID in vivo.

In another embodiment of the invention, the HCS assay provides for a high throughput assay. Preferably, the assay provides automated screening of thousands of test compounds. Compounds tested in the screening method of the present invention are not limited to the specific type of the compound. Non-limiting examples of potential test compounds include chemical agents (such as toxins), pharmaceuticals, peptides, proteins (such as antibodies, cytokines, enzymes, etc.), and nucleic acids, including gene medicines and introduced genes, which may encode therapeutic agents such as proteins, antisense agents (i.e. nucleic acids comprising a sequence complementary to a target RNA expressed in a target cell type, such as RNAi or siRNA), ribozymes, etc. Additionally or alternatively, the assay of the invention may screen a physical agent such as radiation (e.g. ionizing radiation, UV-light or heat); these can be tested alone or in combination with chemical and other agents. In one embodiment, entire compound libraries are screened. Compound libraries are a large collection of stored compounds utilized for high throughput screening. Compounds in a compound library can have no relation to one another, or alternatively have a common characteristic. For example, a hypothetical compound library may contain all known compounds known to bind to a specific binding region. As would be understood by one skilled in the art, the methods of the invention are not limited to the types of compound libraries screened. Non-limiting examples of compound libraries include the sets from LOPAC, Chembridge, Maybridge, LifeChemicals and the NIH Clinical Collection.

In one embodiment, the assay of the invention may also be used to test delivery vehicles. These may be of any form, from conventional pharmaceutical formulations, to gene delivery vehicles. For example, the assay may be used to compare the effects of the same compound administered by two or more different delivery systems (e.g. a depot formulation and a controlled release formulation). It may also be used to investigate whether a particular vehicle could have effects of itself on AID nuclear import. As the use of gene-based therapeutics increases, the safety issues associated with the various possible delivery systems become increasingly important. Thus the models of the present invention may be used to investigate the properties of delivery systems for nucleic acid therapeutics, such as naked DNA or RNA, viral vectors (e.g. retroviral or adenoviral vectors), liposomes, etc. Thus the test compound may be a delivery vehicle of any appropriate type with or without any associated therapeutic agent.

In one embodiment, compounds are evaluated alone. In another embodiment, compounds are evaluated when delivered along with a delivery vehicle. Non-limiting examples of delivery vehicles include polymersomes, vesicles, micelles, plasmid vectors, viral vectors, and the like. As described elsewhere herein, compounds are evaluated for their ability to inhibit the nuclear import of AID. In another embodiment, the methods of the invention comprise selecting a compound that inhibits the nuclear import of AID from a compound library. In another embodiment, test compounds are delivered along with known chemotherapeutic agents to determine whether the test compounds exhibit interference or synergy with other agents.

The test compound may be added to the assay to be tested by any suitable means. For example, the test compound may be injected into the cells of the assay, or it can be added to the nutrient medium and allowed to diffuse into the cells. The assay is also suitable for testing the effects of physical agents such as ionizing radiation, UV-light or heat alone or in combination with chemical agents (for example, in photodynamic therapy).

In situations where “high-throughput” modalities are preferred, it is typical to that new chemical entities with useful properties are generated by identifying a chemical compound (called a “hit compound”) with some desirable property or activity, and evaluating the property of those compounds. A non-limiting example of a high-throughput screening assay is to array the membrane of the invention to 96, 385, 1536, etc. well or slot format to enable a full high throughput screen.

In one embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “hit compounds” or can themselves be used as potential or actual therapeutics. As further discussed below, in one embodiment, the screen and method of the present invention comprise a primary screen, one or more counter screens, and one or more secondary screens. In one embodiment, one or more of the primary screen, counter screens, and secondary screens is a high throughput screen or high content screen, as described elsewhere herein.

### Primary Screen

The system and methods of the invention is based upon the detection of the localization of AID in a living cell or fixed cell. In one embodiment, the system and methods of the invention comprise a primary screen. In one embodiment, the primary screen comprises the acquisition of images of cells to detect AID localization. Localization of AID is made through the detection of a signal corresponding to AID. In one embodiment, the screen of the invention comprises the use of cells that do not natively express AID. In one embodiment, cells are genetically modified to express AID. The present invention is not limited to cells expressing full-length AID protein. One skilled in the art would appreciate the screen of the present invention can use cells which are modified to express only a specific region or regions of AID, for example a fragment of AID containing the amino acid sequences sufficient for nuclear import. In one embodiment, cells of the screen express AID protein that is tagged with a detectable marker, for example fluorescently tagged AID. Non-limiting examples of fluorescent tags include green fluorescent protein (GFP), cyan fluorescent protein (CFP), yellow fluorescent protein (YFP), red fluorescent protein (RFP), orange fluorescent protein (OFP), eGFP, mCherry, hrGFP, hrGFPII, Alexa 488, Alexa 594, and the like. Fluorescent tags may also be photoconvertable such as for example kindling red fluorescent protein (KFP-red), PS-CFP2, Dendra2, CoralHue Kaede and CoralHue Kikume. However, the invention should not be limited to a particular label. Rather, any detectable label can be used to tag AID.

In one embodiment, the screen comprises a cell or cell population modified to express AID and/or other proteins of interest. In one embodiment, the cell or cell population is modified by administering an expression vector encoding the protein of interest. As would be understood by those skilled in the art, the expression vector used to modify the cell or cell population of the screen includes any vector known in the art such as cosmids, plasmids, phagemid, lentiviral vectors, adenoviral vectors, retroviral vectors, adeno-associated vectors, and the like.

The expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used. In one embodiment, the cell or cell population of the screen are administered a lentiviral vector encoding AID. For example, in one embodiment, the cell or cell population of the screen are administered a pLVTHM lentiviral plasmid comprising a nucleic acid sequence encoding AID.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Employing genetic engineering technology necessarily requires growing recombinant host cells (e.g., transfectants, transformants) under a variety of specified conditions as determined by the requirements of the cells and the particular cellular state desired by the practitioner. In one embodiment, genetic engineering includes transiently transfected cells or the establishment of stable expression cell lines. For example, a host cell may possess (as determined by its genetic disposition) certain nutritional requirements, or a particular resistance or sensitivity to physical (e.g., temperature) and/or chemical (e.g., antibiotic) conditions. In addition, specific culture conditions may be necessary to regulate the expression of a desired gene (e.g., the use of inducible promoters), or to initiate a particular cell state (e.g., yeast cell mating or sporulation). These varied conditions and the requirements to satisfy such conditions are understood and appreciated by practitioners in the art.

The recombinant vectors harboring the sequence encoding AID, or other elements of the present invention, can be introduced into an appropriate host cell by any means known in the art. For example, the vector can be transfected into the host cell by calcium phosphate co-precipitation, by conventional mechanical procedures such as microinjection or electroporation, by insertion of a plasmid encased in liposomes, and by virus vectors. These techniques are all well-known and routinely practiced in the art, e.g., Brent et al., Current Protocols in Molecular Biology, John Wiley & Sons, Inc. (ringbou ed., 2003); and Weissbach & Weissbach, Methods for Plant Molecular Biology, Academic Press, NY, Section VIII, pp. 42 1-463, 1988. Host cells which harbor the transfected recombinant vector can be identified and isolated using the selection marker present on the vector. Large numbers of recipient cells may then be grown in a medium which selects for vector-containing cells. These cells may be used directly or the expressed recombinant protein may be purified in accordance with conventional methods such as extraction, precipitation, chromatography, affinity methods, electrophoresis and the like. The exact procedure used will depend upon the specific protein produced and the specific vector/host expression system utilized.

In an embodiment, host cells for expressing the recombinant vectors are eukaryotic cells. Eukaryotic vector/host systems, and mammalian expression systems, allow for proper post-translational modifications of expressed mammalian proteins to occur, e.g., proper processing of the primary transcript, glycosylation, phosphorylation and advantageously secretion of expressed product. Therefore, eukaryotic cells such as mammalian cells can be the host cells for the protein of a polypeptide of interest. Examples of such host cell lines include CHO, BHK, HEK293, VERO, HeLa, COS, MDCK, NS0 and W138. Such cells lines can be transiently transfected with AID and/or other elements of the invention. Alternatively, stable cell lines genetically altered to constitutively express AID and/or other elements of the invention can be generated by methods known in the art.

In some embodiments, engineered mammalian cell systems that utilize recombinant viruses or viral elements to direct expression of the protein of interest are employed. For example, when using adenovirus expression vectors, the coding sequence of AID or other protein of interest may be ligated to an adenovirus transcription/translation control complex, e.g., the late promoter and tripartite leader sequence. This chimeric gene may then be inserted into the adenovirus genome by in vitro or in vivo recombination. Insertion in a non-essential region of the viral genome (e.g., region E1 or E3) will result in a recombinant virus that is viable and capable of expressing the polypeptide of interest in infected hosts (e.g., see Logan & Shenk, 1984 Proc. Natl. Acad. Sci. USA 81:3655-3659). Alternatively, the vaccinia virus 7.5K promoter may be used. (e.g., see, Mackett et al., 1982, Proc. Natl. Acad. Sci. USA, 79:7415-7419; Mackett et al., 1984, J. Virol. 49:857-864; Panicali et al., 1982, Proc. Natl. Acad. Sci. USA, 79:4927-4931). In certain embodiments, vectors are based on bovine papilloma virus which has the ability to replicate as extrachromosomal elements (Sarver et al., 1981, Mol. Cell. Biol. 1:486). These vectors can be used for stable expression by including a selectable marker in the plasmid, such as the neo gene. Alternatively, the retroviral genome can be modified for use as a vector capable of introducing and directing the expression of the gene of interest in host cells (Cone & Mulligan, 1984, Proc. Natl. Acad. Sci. USA 8 1:6349-6353). High level expression may also be achieved using inducible promoters, including, but not limited to, the metallothionine IIA promoter and heat shock promoters.

In one embodiment, the cells of the screen are modified to transiently express AID. In another embodiment, the cells of the screen are modified for the stable expression of AID. For example, in one embodiment, a cell line which stably expresses AID, is generated and maintained under standard culturing protocols known in the art. In one embodiment, a cell of the screen comprises a nucleic acid encoding AID

The present invention is related to screening methods comprising the automated detection of the cellular localization of proteins. In one embodiment, the localization of AID, or other elements of interest, is determined from images taken of cells expressing AID, or other elements of interest. The localization of AID, or other elements of interest, may be determined in the live cell of the assay, or alternatively after the cell has been fixed. The present invention is not limited to the type or mode of microscopy utilized in imaging of the cells of the screen. In one embodiment, acquired images obtained through standard fluorescent microscopy techniques known in the art, detects the localization of the fluorescent signal in a cell, thereby detecting the localization of AID within a cell. It is known in the art that in control conditions, AID shuttles into the nucleus and nuclear export of AID occurs at a fast rate giving rise to the fact that most AID is found in the cytoplasm. Thus, in these conditions, images of the cell would demonstrate AID localization in the cytoplasm. Therefore, in embodiments wherein cells express fluorescently tagged AID, images of the cells exhibit a bright cytoplasm and a dark nucleus. As would be understood by those skilled in the art, full length AID protein, or portions thereof, can be used in the screening methods of the invention. For example, in one embodiment, cells of the screen comprise full length AID. In another embodiment, cells of the invention comprise only specific regions of AID known to influence cellular localization.

In one embodiment, the primary screen of the invention comprises the step of adding a compound known to inhibit AID nuclear export. An exemplary compound that inhibits AID nuclear export, and that can be used in the methods of the invention, is Leptomycin B (LMB). Another exemplary nuclear export inhibitor, useful in the present invention is Ratjadone A, which is chemically related to LMB. While the described screen is exemplified herein with the use of LMB, one skilled in the art would recognize that any other known nuclear export inhibitor (e.g. Ratjadone A) is similarly useful in the screen of the invention.

Application of LMB to the cell inhibits nuclear export, thereby producing a build-up of AID in the nucleus. In embodiments wherein cells express fluorescently tagged AID, images of LMB treated cells appear as a dark cytoplasm and a bright nucleus. The fast shuttling of AID out of the nucleus provides for the primarily cytoplasmic localization of AID in basal conditions which can make detection of on target effects of changes in AID nuclear import afforded by test compounds difficult to observe. Use of nuclear export inhibitors (e.g. LMB) in the screen allows for easier detection of on target effects of changes in AID nuclear import caused by test compounds.

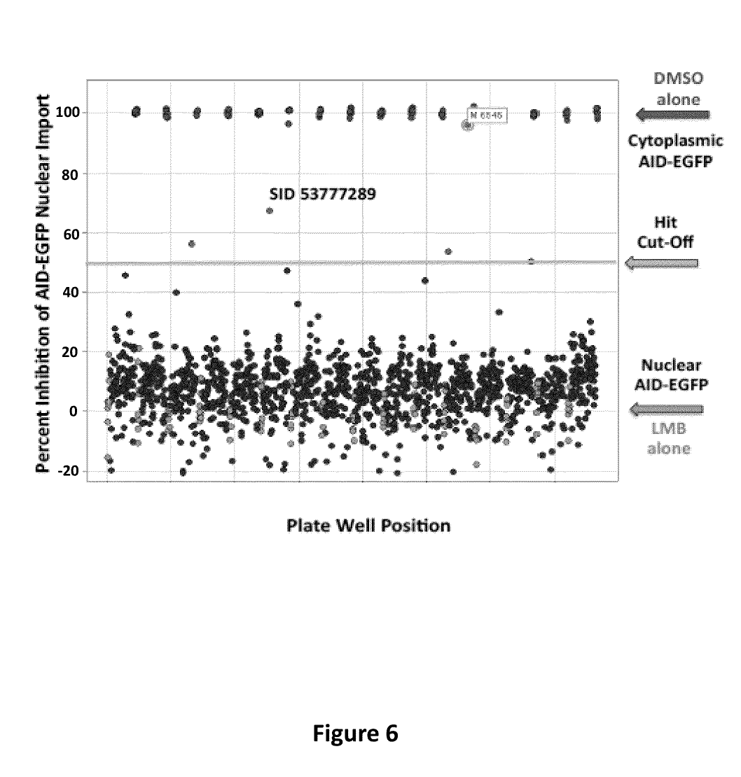

In one embodiment, localization of AID is quantitatively determined by the automated calculation of the proportion of AID in nuclear to cytoplasmic compartments, referred to as the AID N/C ratio. In one embodiment, AID in each compartment is determined by quantifying the fluorescence due to fluorescently tagged AID in each compartment. In control conditions (e.g., in the absence of LMB), untreated cells (or vehicle treated) have a relative low N/C ratio. Cells treated with LMB would have a relative high N/C ratio. These two conditions define the lower and upper limits of the AID N/C ratio. Test compounds that inhibit AID nuclear import, and therefore designated as hits, have AID N/C ratio close to the lower limit. In certain embodiments, AID cytoplasmic localization in untreated and/or vehicle treated cells is denoted as 100% and AID nuclear localization in LMB alone treated cells is denoted as 0% (FIG. 6). In one embodiment, hits are defined as those test compounds that inhibit AID nuclear import by greater than 90%. In another embodiment, hits are defined as those test compounds that inhibit AID nuclear import by greater than 80%. In another embodiment, hits are defined as those test compounds that inhibit AID nuclear import by greater than 70%. In another embodiment, hits are defined as those test compounds that inhibit AID nuclear import by greater than 60%. In another embodiment, hits are defined as those test compounds that inhibit AID nuclear import by greater than 50%. In another embodiment, hits are defined as those test compounds that inhibit AID nuclear import by greater than 40%. In another embodiment, hits are defined as those test compounds that inhibit AID nuclear import by greater than 30%. In another embodiment, hits are defined as those test compounds that inhibit AID nuclear import by greater than 20%. In another embodiment, hits are defined as those test compounds that inhibit AID nuclear import by greater than 10%.

The systems and methods of the invention comprise the steps of delivering a test compound to a cell expressing AID and observing the localization of AID in response to the compound. In another embodiment, the present invention comprises delivering a test compound and LMB to a cell expressing AID, and observing the localization of AID in response to the test compound in the presence of LMB. In one embodiment, LMB is administered to a cell after a defined time period following administration of a test compound. For example, in one embodiment, a test compound is administered to a cell and the cell is in the presence of the test compound for a given period of time prior to the administration of LMB. In one embodiment, a test compound is administered to a cell and the cell is in the presence of the test compound for 0-48 hours prior to LMB administration. In another embodiment, a test compound is administered to a cell and the cell is in the presence of the test compound for 3-24 hours prior to LMB administration. In another embodiment, a test compound is administered to a cell and the cell is in the presence of the test compound for 6-12 hours prior to LMB administration. In one embodiment, LMB is administered to the cell and the cell is in the presence of LMB for 0-48 hours. In another embodiment, LMB is administered to the cell and the cell is in the presence of LMB for 3-24 hours. LMB is administered to the cell and the cell is in the presence of LMB for 6-12 hours.

LMB is administered to a cell at a concentration known to inhibit nuclear export, for example nuclear export of AID. In one embodiment, LMB is administered to the cell at a concentration of 1-10,000 ng/mL. In another embodiment, LMB is administered to the cell at a concentration of 10-1,000 ng/mL. In another embodiment, LMB is administered to the cell at a concentration of 50-500 ng/mL. In another embodiment, LMB is administered to the cell at a concentration of 100-200 ng/mL.

A test compound can be identified as a potential inhibitor of AID nuclear import if the test compound reduces the N/C ratio compared to the N/C ratio resulting from the LMB application alone in a control cell. LMB application alone defines the maximum N/C ratio, while untreated negative control defines the minimum N/C ratio. The screening of compounds with and without the presence of LMB produces an assay designed to emphasize compound effects on AID nuclear import. Further, the controls with and without LMB define the dynamic range of the assay signal window and enable quality control metrics including z′-factor coefficient, signal to background ratios and coefficient of variance of the controls to be determined and measured.

In one embodiment, the primary screen comprises an internal counter screen. In one embodiment, the primary screen of the present invention comprises cells expressing AID, or a portion thereof, and a non-AID element whose nuclear import is distinct from that of AID, thereby serving as a co-expressed counter screen. In one embodiment, the non-AID element is a nuclear localized element. In one embodiment, the element of the internal counter screen of the primary screen is the nuclear localized transaldolase 1 (TALDO1). In one embodiment, the nuclear localized element is histone H1.

In some instances, the use of TALDO1 as the internal or simultaneous counter screen is preferred as it uses the same transporters as AID. Thus, compounds that affect AID nuclear import and not TALDO1 have the highest probability of reacting with or binding to AID, rather than the transporter, nuclear pore complex, or anything else common to the import mechanism of TALDO1. Thus, compounds identified in an AID/TALDO1 primary/counter screen have the highest probability of being AID-specific nuclear import inhibitors, and therefore are least likely to have off-target and/or toxic effects.

In one embodiment, AID and the nuclear localized element are each labeled with a different detectable marker, and images of cells are used to detect the localization of both AID and the nuclear localized element. In one embodiment, AID and the nuclear localized element are each labeled with a differently colored fluorescent marker. In one embodiment, cells expressing AID and a nuclear localized element are treated with a test compound, and the localization of AID and the nuclear localized element is observed in response to the test compound. In another embodiment, cells expressing AID and a nuclear localized element are treated with a test compound in the presence of LMB, and the localization of AID and nuclear localized element is observed in response to the test compound in the presence of LMB. In this instance, a test compound is identified as a potential specific and selective inhibitor of AID nuclear import if application of the test compound in the presence of LMB reduced the N/C ratio for AID but does not change the N/C ratio for the nuclear localized element compared to LMB application in the absence of the test compound.

In another embodiment, the element of the internal counter screen of the primary screen is a cytoplasmic localized protein, for example the cytoplasmic localized APOBEC1 complementation factor (ACF). In yet another embodiment, the element of the internal counter screen of the primary screen is the nuclear localized heterogeneous nuclear ribonucleoprotein A1 (hnRNP A1). As would be understood by those skilled in the art, elements of the internal counter screen are not limited by those listed herein. Rather, any element whose nuclear and cytoplasmic distribution can be observed may be used in the internal counter screen.

HCS assays typically comprise automated screening techniques to generate a high level of information from an experiment. In one embodiment, the system of the invention comprises numerous test compounds screened on cells cultured on a multi-well plate. Non-limiting examples of multi-well plates include a 6-well plate, a 24-well plate, a 96-well plate, and a 384-well plate. As such, each well comprises its own individual experiment detecting the response to a single test compound. In one embodiment, LMB treated alone and untreated or vehicle treated negative controls are conducted on each multi-well plate. Statistical analysis performed on the control wells enable the determination of the overall quality of experimentation done on the entire plate. In plates with controls determined to pass a statistical standard, test compounds that reduce N/C ratio of AID by a pre-defined amount relative to the mean of all compounds tested on the plate, that are not acutely cytotoxic and/or fluorescent outliers are flagged as “hits” as inhibitors of AID nuclear import. As such, the primary screen of the invention narrows a first population of test compounds into a second, smaller, population of test compounds that retain the ability to inhibit AID nuclear import.

### Counter Screens

In one embodiment, the system and methods of the invention comprise one or more counter screens that accompany the primary screen. In one embodiment, a counter screen is used to evaluate a population of test compounds, identified by the primary screen as being inhibitors of nuclear import of AID, for being specific and selective inhibitors of AID nuclear import. The methods of the present invention are directed towards a high throughput screen that identifies an inhibitor of nuclear import that is specific for AID. Compounds of greatest clinical benefit are those that inhibit AID nuclear import, but do not affect the nuclear import of other proteins, thus minimizing off target side effects. Counter screens of the invention are used to evaluate whether test compounds that alter AID cellular localization do so by some shared mechanism that would alter cellular localization of non-AID proteins. An example of such a shared mechanism includes binding to shared import machinery.

In one embodiment, the counter screen comprises an additional HCS assay. Therefore, as would be understood by those skilled in the art, the counter screens of the invention are not limited in the types of cells used or in the methods and techniques used to express or detect proteins of interest, as described elsewhere herein. In one embodiment, the screen comprises the addition of a test compound from the hits identified in the primary screen to a cell expressing a predominately cytoplasmic element that is not AID but is known to shuttle between the cytoplasm and nucleus (i.e. non-AID protein or non-AID shuttling element) and observing the localization of the non-AID shuttling protein in response to the compound. As described elsewhere herein, cells of the assay can be of any type so long as they express the protein of interest. In one embodiment, the non-AID shuttling element is labeled with a detectable marker. In one embodiment, the detectable marker is a fluorescent tag. As described elsewhere herein, the fluorescent tag may be of any tag known in the art.

In one embodiment, the counter screen of the invention comprises the addition of a test compound from the hits identified in the primary screen and LMB to a cell expressing the non-AID shuttling element, and observing the localization of the non-AID shuttling element in response to the addition of the test compound and LMB. Therefore, in this instance, the counter screen identifies non-AID specific effects of the test compound. As described elsewhere herein, in certain embodiments, the test compound is administered for a defined amount of time prior to administration of LMB. As such, test compounds from the hits identified in the primary screen, that do not alter the N/C ratio of the non-AID shuttling element in a test cell treated with the test compound and LMB, compared to the N/C ratio in a control cell treated with only LMB, are identified as AID specific nuclear import inhibitors.

In another embodiment, the counter screen of the invention comprises the addition of a test compound from the hits identified in the primary screen, with or without the addition of LMB, to a cell expressing a predominately nuclear localized element, and observing the localization of the nuclear localized element in response to the test compound. In one embodiment the nuclear localized element becomes localized in the cytoplasm in cytotoxic conditions. Therefore, in this instance, the counter screen determines the cytotoxicity of the test compound. As such, test compounds from the hits identified in the primary screen, that do not alter the N/C ratio of the nuclear localized element in a test cell treated with the test compound and LMB, compared to the N/C ratio in a control cell treated with only LMB, are designated for further study. In one embodiment, the nuclear localized element is the nuclear localized protein, A1

In one embodiment of the counter screen, the cells of the screen express the non-AID shuttling element and AID, each with a different detectable marker. In one embodiment, the counter screen comprises the addition of a test compound from the hits identified in the primary screen and LMB to a cell expressing AID and the non-AID shuttling element, and observing the localization of both AID and the non-AID shuttling element in response to the addition of the test compound and LMB. Therefore, in this instance, the counter screen identifies non-AID specific effects of the test compound. As such, test compounds from the hits identified in the primary screen, that do not alter the N/C ratio of the non-AID shuttling element but reduces the N/C ratio of AID compared to addition of LMB alone, are identified as AID specific nuclear import inhibitors.

As would be understood by those skilled in the art, the counter screen of the invention is not limited to the identity of the non-AID shuttling element. Rather, any element that under normal conditions, shuttles in and out of the nucleus and uses the same import machinery, can be used as the non-AID shuttling element. Such elements are used to evaluate if test compounds only affect the AID N/C ratio. In one embodiment, the non-AID shuttling element does not share nuclear transporters with AID, for example ACF.

In another embodiment the non-AID shuttling element shares the same nuclear transport machinery, for example transaldolase 1 (TALDO-1). In this instance, the counter screen evaluates test compounds for the ability to inhibit AID, but not TALDO-1, thereby identifying compounds that bind to AID and not the nuclear import machinery.

In one embodiment, as described elsewhere herein, the counter screens of the invention are HCS assays. As would be understood by those skilled in the art, multiple counter screens can be run in parallel. Further, as described elsewhere herein, HCS assays are performed on multi-well plates, with appropriate positive and negative controls, providing a statistical basis for the inclusion or exclusion of data obtained from the given plate.

In one embodiment of the present invention, non-AID shuttling elements are included in the primary screen of the invention as a built-in and simultaneous counter screen. Thus, as would be understood by those skilled in the art, all elements described herein as elements of the counter screens of the invention are all able to be incorporated into the primary screen of the invention. Therefore, in one embodiment, the invention comprises only a primary screen that can identify an AID nuclear import inhibitor and evaluate the specificity and toxicity of the inhibitor in a single screen. Further, primary and counter screens of the invention can be run sequentially or alternatively in parallel so as to reduce the total time required to screen all potential test compounds.

### Secondary Assays

The present invention is directed towards methods of identifying AID specific nuclear import inhibitors. In one aspect of the invention, the methods comprise secondary assays of compounds identified in the primary and counter screens of the invention, described elsewhere herein. Such secondary assays evaluate the potential of identified compounds to inhibit cellular processes that are downstream and dependent on AID nuclear localization. Such processes include but are not limited to SHM and CSR.

AID nuclear import and deaminase activity on genomic DNA are required for both somatic hypermutation (SHM) and class switch recombination (CSR). In one embodiment, the secondary assay of the invention comprises SHM analysis. In one embodiment of this analysis, cells express an impaired gene encoding a marker (e.g. GFP, mCherry, His-tag, and the like), wherein the gene is under control of a CMV promoter and can be reactivated by AID mutator activity. Compounds identified in the primary and counter screens of the invention are used in this assay to evaluate their potential to inhibit AID mutator activity. In this assay, successful compounds are those that reduce the production of the marker, compared to conditions when the compound is absent.

In one embodiment, the secondary assay of the invention comprises a CSR assay. In one embodiment, the CSR assay comprises IgM+ B cells stimulated to induce proliferation and class switching by adding to the cells compounds such as anti-CD40 and IL-4. The assay further comprises detecting the generation of IgG+ cells in the days following stimulation by, for example, flow cytometry and ELISA. Compounds identified in the primary and counter screens of the invention are used in this assay to evaluate their potential to inhibit AID mediated class switching. In this assay, successful compounds are those that reduce the production of IgG+ cells, compared to conditions when the compound is absent.

As would be understood by those in the art, the secondary assays used in the present invention are not limited to the SHM and CSR assays described herein. Rather, any secondary assay known in the art that can quantitatively or qualitatively measure AID mutagenic activity may be used to evaluate the functional activity of compounds identified in the primary and secondary screens of the invention.

### Therapeutics

Selective inhibitors of AID nuclear import that are identified by the systems and methods of the invention are innovative for therapeutics because these compounds have fewer toxic side effects than targeting either AID expression through transcription inhibitors (e.g. NFκB inhibitors), AID phosphorylation (e.g. PKA inhibitors), chaperones involved in AID shuttling or AID cytoplasmic retention (e.g. Hsp90). Limiting AID activity by reducing its nuclear import is less toxic than developing cytidine deaminase inhibitors because the deaminase domain is homologous to metabolic deaminases. Restriction of AID to the cytoplasm is not toxic because AID has no other known functions and does not have a mitochondrial import signal. Further, AID-selective nuclear import inhibitors are unlikely to immunocompromise patients as do radiation, DNA damaging chemotherapeutics or the B cell depleting agent Rituximab (an FDA-approved drug for B cell malignancies and rheumatoid arthritis and used off label in other autoimmune diseases).

Without wishing to be bound by any particular theory, it is believed that antibody expression from the existing repertoire of cell immunoglobulin genes will be unaffected by AID-selective nuclear import inhibitors and only the production of novel antibodies to new antigens will be impacted. AID activity required for antibody diversification during a de novo immune response could be regulated by dose and frequency of dosing to accommodate individual patient needs for adaptive immunity (e.g. during flu season) and each patient's unique expression level of AID.

Therefore, the system and methods of the invention provides for the identification or selection of AID specific nuclear import inhibitors from a population of test compounds. In another aspect of the invention, AID specific nuclear import inhibitors identified through the screening methods of the invention are used to treat or prevent various forms of cancer. For example, the identified inhibitors can be used to prevent, delay, or inhibit cancer progression. Non-limiting examples of the forms of cancers that identified compounds would be effective include Follicular Lymphoma, chronic lymphocytic leukemias, acute lymphoblastic leukemias, diffuse large B cell lymphomas, Burkiett's mantle cell lymphomas, Hodgkin's disease, and solid tumors of the gastrointestinal system, urogenital systems, breast, skin, and nervous system.

Constitutive AID expression has been shown in a number of B cell malignancies, including follicular lymphoma (FL), where sustained expression has been suggested to be causal in disease progression by inducing accumulation of oncogene mutations leading to chromosomal translocations (Hardianti et al., 2004, Leukemia, 18(4): 826-831; Smit et al., 2003, Cancer Res, 63(14): 3894-3898). Follicular lymphoma initially has an indolent course, and patients with follicular lymphoma often experience little to no symptoms for many years. However, progression of follicular lymphoma into an advanced stage or even into more aggressive forms of lymphoma, then threatens the life of the patient. Progression of follicular lymphoma is suggested to be mediated by AID activity, through the build-up of AID-induced oncogenic mutations and chromosomal translocations. As discussed herein, an inhibitor identified by the screen of the invention can be used to limit AID access to the genome, thereby preventing or reducing oncogenic mutation. Therefore, an inhibitor identified by the screen of the invention can be used to prevent disease progression following an initial cancer diagnosis. The pristane plasmacytoma mouse model showed that AID expression was haplo-insufficient and heterozygous mutants developed disease at a significantly lower rate than wild-type mice (Takizawa et al., 2008, J Exp Med). Thus even a partial reduction in AID nuclear import will decrease AID's mutagenic, cancer-promoting activity.

Inhibition of AID access to the nucleus and inhibition of AID-induced accumulation of oncogenic mutations is not only applicable to inhibition of progression of Follicular lymphoma. Rather, limiting access of AID to the nucleus, by way of the specific nuclear import inhibitors identified by the screen of the invention can be used to inhibit disease progression in many forms of cancer. In this way, a diagnosed cancer or tumor would be inhibited to progress or transform into a more aggressive form. Elevated expression of AID has also been described in a subset of B cell chronic lymphocytic leukemias (CLL) and acute lymphoblastic leukemias (ALL), most diffuse large B cell lymphomas (DLBCL), some Burkett's mantle cell lymphomas, as well as Hodgkin's disease (Hardianti et al., 2004, Leukemia, 18(4): 826-831; Smit et al., 2003, Cancer Res, 63(14): 3894-3898; Greeve et al, 2003, Blood, 101: 3574-3580; McCarthy et al., 2003, Blood, 101: 4903-4908; Oppezzo et al., 2003, Blood, 101: 4029-4032; Babbage et al., 2004, Blood, 103(7): 2795-2798; Lossos et al., Leukemia, 2004, 18: 1775-1779; Pasqualucci et al., Blood, 2004, 104: 3318-3325; Heintel et al., 2004, Leukemia, 18(4): 756-762; Feldahn et al., 2007, J Exp Med, 204(5): 1157-1166; Deutsch et al., 2007, Blood, 109(8): 3500-3504; Lenz et al., 2007, J Exp Med., 204(3): 633-643; Hardianti et al, 2005, Eur J Haematol, 74(1): 11-19; Epeldegui et al, 2005, Curr Opin Oncol, 18(5): 444-448; Mottok et al., 2005, J Clin Pathol, 58(9): 1002-1004; Greiner et al, 2005, J Pathol, 205(5): 541-547). AID expression has also been detected in non-lymphoid primary tumors, including breast, liver and gastric cancer (Babbage et al, 2006, Cancer Res, 66(8): 3996-4000; Kim et al., 2007, 28(6): 333-339; Kou et al, 2007, Int J Cancer, 120(3): 469-476; Komori et al, 2008, 47(3): 888-896).

AID specific nuclear import inhibitors, identified through the methods of the invention, can be administered to a subject or patient through any means known in the art. Administration of the therapeutic agent in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art

As contemplated elsewhere herein, inhibitors identified through methods of the invention may comprise nucleic acids, including DNA and RNA sequences. Pharmaceutical formulations, dosages and routes of administration for nucleic acids are generally disclosed, for example, in Felgner et al., 1987. Further, administration of proteins, peptides, siRNA and other compositions that display therapeutic benefit may be accomplished through administration of nucleic acid molecules that encode for such compositions (see, for example, Felgner et al. 1987, U.S. Pat. No. 5,580,859, Pardoll et al. 1995; Stevenson et al. 1995; Molling 1997; Donnelly et al. 1995; Yang et al. II; Abdallah et al. 1995)

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent may be directly injected into the tumor. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The expression vectors, transduced cells, polynucleotides and polypeptides (active ingredients) of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly(lactic acid) or ethylenevinylacetate copolymers. In addition to being incorporated, these agents can also be used to trap the compound in microcapsules.

Accordingly, the pharmaceutical composition of the present invention may be delivered via various routes and to various sites in a mammal body to achieve a particular effect. One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Establishing the Cell-Based, Fluorescent, Primary Assay for HCS with Simultaneous Counter Screen

A cell-based assay for HCS is developed in which AID (a predominantly cytoplasmic DNA-binding protein that shuttles from the cytoplasm to the nucleus) and Histone H1 (a non-shuttling nuclear DNA-binding protein) are coexpressed as a primary screen for AID nuclear import inhibitors. A non-limiting example of such an assay comprises labeled AID and Histone H1 such as AID-EGFP and mCherry Histone H1). In another embodiment, of the screen, the assay comprises cells co-expressing AID and TALDO, as described elsewhere herein. In any event, the assay of the invention comprises a cell or cell population used for a primary screen for inhibitors of AID nuclear import and simultaneously be used to counter screen for off-target effects on nuclear protein import and cytotoxicity, respectively. Preliminary data show assay feasibility with appropriate uniformity and signal to noise ratios that enable threshold for nuclear/cytoplasmic protein distributions that is set by computational image analysis software. Hits from the primary screen is selected for further analysis if they reduce the nuclear to cytoplasmic (N/C) ratio of AID but do not affect H1 nuclear retention.

### Primary Assay

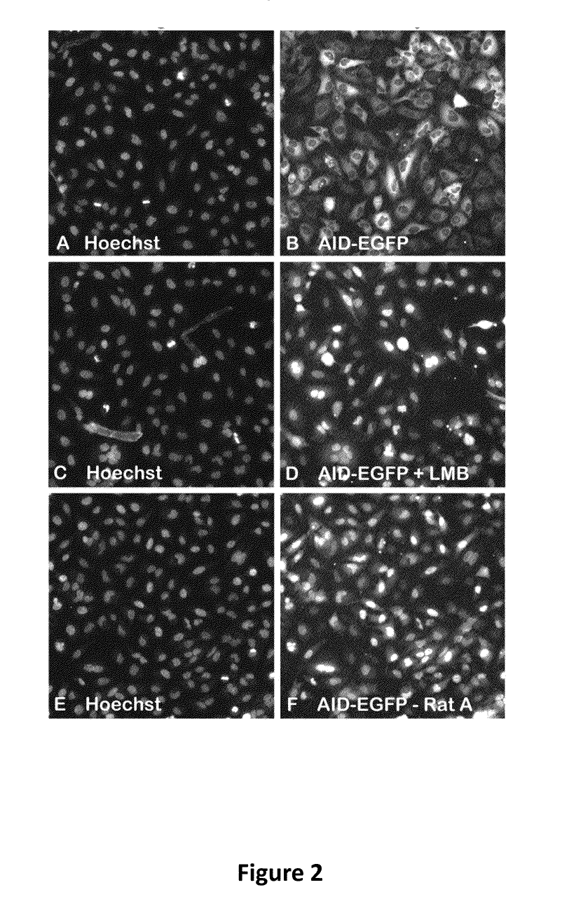

HCS assay development involves the selection of the most appropriate cellular model (Dudgeon et al., 2010, J. Biomol. Screen, 15: 766-782; Nickischer et al., 2006, Methods Enzymol, 414: 389-418; Williams et al., 2006, Methods Enzymol, 414: 364-89). In this regard, AID shuttling is not specific to B cells, making it possible to use a variety of cell types to study AID structure and function (Okazaki et al., 2002, Nature, 416: 340-5; Yoshikawa et al., 2002 Science, 296: 2033-6). However, the ability of LMB to induce quantitative AID nuclear retention was more uniform in HeLa cells (a finding has been independently confirmed by comparing the AID responses to LMB in transiently transfected HeLa, HEK293T, U2OS, A549, CaCo2, HepG2 and CHO cells). It is generally known in the AID field that stable cell lines expressing high levels of AID are difficult to maintain and, in some cell types, long-term expression of recombinant proteins has been shown to diminish due to silencing of the CMV. Consequently, AID-EGFP was subcloned from a pIRES vector into the lentiviral vector pLVTHM (Addgene) using the EF1-α promoter to drive moderate to low level expression. pLV-AID-EGFP psuedotyped virus was transduced into HeLa S3 cells (ATCC) that were live-cell flow sorted to obtain a homogenous population of fluorescent cells. Subcloning of a cell line was not thought to be necessary given the ability to threshold fluorescence in individual cells during high content screening. These cells have been maintained in culture with little or no loss of fluorescence for 2 months. HeLa AID-EGFP cells plated into 384-well plates at varying cell densities (1,250 to 10,000 cells per well) were simultaneously fixed with formaldehyde and counter stained with Hoechst and then imaged using both the IXU Ultra and ArrayScan VTI platforms with 10× and 20× objectives. Image quantification was performed with the multi-wavelength cell scoring (MWCS) & translocation enhanced (TE) image analysis modules and the molecular translocation (MT) image algorithm for the IXU Ultra and ArrayScan VTI, respectively. Based on image acquisition, 2,500 cells per well was determined ideal for either platform regardless of the objective. All cells revealed predominant cytoplasmic AID-EGFP fluorescence with slight to no nuclear fluorescence (FIG. 2A, FIG. 2B). To establish the LMB response within the context of an HCS system and assess the assay parameters, cells were treated in 384-well format with varying doses (0.2 to 200 ng/ml) of either LMB or Ratjadone A (chemically related to LMB) for varying durations up to 200 min. The cells demonstrated maximal nuclear retention of AID-EGFP when treated with 100 ng/ml either LMB or Ratjadone A for 180 min (FIG. 2C through FIG. 2F). There was a 4.5-fold difference between the nuclear to cytoplasmic (N/C) fluorescence ratio of LMB-treated cells and cells only treated with the solvent for LMB (0.5% methanol). The assay had a Z′-factor of 0.52 and a CV of 0.11

Histone H1 is an excellent choice as an effective internal counter screen in the primary HCS assay because it binds to genomic DNA but does not shuttle following its initial nuclear import. Moreover, H1 nuclear localization is essential for chromatin integrity and function and as such, serves as a reporter for nuclear stability and cell health. Compounds that inhibit AID-EGFP nuclear import (N/C ratio) by ≧three standard deviations below the mean of the LMB treated control plate N/C ratio (z-score ≦−3) is flagged as a hit as long as they do not alter H1 nuclear distribution.

The innovative design of the assay for the primary screen lies in its ability to focus the search for compounds that affect the nuclear import pathway because LMB will be used to block nuclear export. The assay development includes optimizing the nuclear retention of EGFP-AID in response to LMB by evaluating different expression levels of EGFP-AID and optimizing the duration and concentration of LMB treatment and different sources of LMB. The fully developed and optimized AID nuclear import and retention assay is sufficiently robust and reproducible for HCS with favorable statistical performance indices including z′ factor, signal to noise and signal to background ratios and low CVs.

The screened library may contain compounds that: (i) inhibit CRM1-dependent nuclear export of AID, (ii) enhance nuclear import of AID or (iii) enhance AID nuclear export. However compounds that inhibit CRM1-dependent nuclear export of AID or enhance nuclear import of AID would lead to high N/C ratios that would not be selected as hits. It is acknowledged that given that both arms of the shuttling pathway affect AID localization, it may be difficult to distinguish the effects of nuclear import inhibitors from nuclear export activators because both would lead to predominantly cytoplasmic AID. Export activators would be false positives because cells treated with these compounds would have a reduced N/C ratio of fluorescent AID upon LMB treatment. The potential that these compounds will be included in the initial hits will depend on: (i) their mechanism of action relative to CRM1 (simulating CRM1 binding to cargo or stimulating Ran GTPase) and (ii) their potency as enhancers of AID nuclear export relative to LMB. If these compounds act on CRM1 they will be identified in the ACF/hnRNP A1 counter screen as reduced ACF N/C ratio and thus these compounds would not be further assessed. It is also important to keep in mind that nuclear import inhibitors prevent AID access to the genome but AID nuclear export enhancers while reducing nuclear abundance of AID may still allow AID access to the genome. This nuance may make nuclear import inhibitors more effective as inhibitors of SHM and CSR but CSR (which depends on shuttling) may be stimulated by nuclear export activators and on this basis, these compounds can be distinguished.

### Example 2

### Develop and Optimize HCS Assay Parameters

HCS assay development involves the optimization of sample preparation methods, image acquisition procedures, and image analysis algorithms. HCS sample preparation is a complex, multi-component process that includes selection and optimization of the cell line, microtiter plate, fixative, blocking buffer, wash buffer, primary and secondary antibodies, and fluorescent probes (Nickischer et al., 2006, Methods Enzymol, 414: 389-418; Williams et al., 2006, Methods Enzymol, 414: 364-89; Dudgeon et al., 2010, Drug Dev Technol., 8: 437-458; Gough et al., 2006, Methods in Molecular Biology, 356: 41-61; Johnston et al., 2008, Assay Drug Dev Technol, 6: 505-518; Trask et al., 2006, Methods Enzymol, 414: 419-39; Trask et al., 2009, Methods Mol Biol, 2009. 565: 159-86). The number and types of fluorescent probes determine how many channels need to be acquired and which excitation and emission filters to use (Gough et al., 2006, Methods in Molecular Biology, 356: 41-61; Johnston et al., 2008, Assay Drug Dev Technol, 6: 505-518). The sample quality and optical resolution required influence the selection of the objective and exposure times required for image acquisition (Gough et al., 2006, Methods in Molecular Biology, 356: 41-61; Johnston et al., 2008, Assay Drug Dev Technol, 6: 505-518). The cell seeding density determines the number of image fields that need to be acquired (Gough et al., 2006, Methods in Molecular Biology, 356: 41-61; Johnston et al., 2008, Assay Drug Dev Technol, 6: 505-518).

HEK293T cells are transfected by adapting and optimizing the protocol described elsewhere herein. Greiner 384-well black walled clear bottomed plates that are either not coated or have been pre-coated with collagen, poly-D-lysine or poly-L-lysine are evaluated to determine which plates provide the best surface for the EGFP-AID/mCherry-H1 cell line to withstand the rigors of the automated HCS assay procedure. LMB concentration response and time course experiments are conducted to determine which treatment conditions provide the largest and most reproducible EGFP-AID N/C ratio, largest signal window and z′ factor. The selected cell count parameter from the image analysis algorithm and the EGFP-AID N/C ratio are used to evaluate the variability in the data. Additional EGFP-AID/mCherry-H1 cell seeding density experiments in the selected plates are performed to identify the optimal cell density that provides the largest and most reproducible EGFP-AID nuclear to cytoplasm ratio/difference assay signal window and z′ factors with the lowest cell culture burden. Since compound libraries are dissolved in DMSO, the amount of solvent that the cells can tolerate without altering the assay signal window is evaluated. Cell morphology changes that affect nuclear swelling or shrinkage can affect the nuclear boundaries and thereby the quantitation. To determine DMSO tolerance, the maximum amount of solvent that does not irreversibly affect the EGFP-AID nuclear to cytoplasm ratio/difference assay signal window and z′ factors is determined Plates of cells are treated with control solvents or library compounds followed by LMB as described in elsewhere herein, simultaneously fixed and stained with Hoechst and processed for image analysis and quantification.

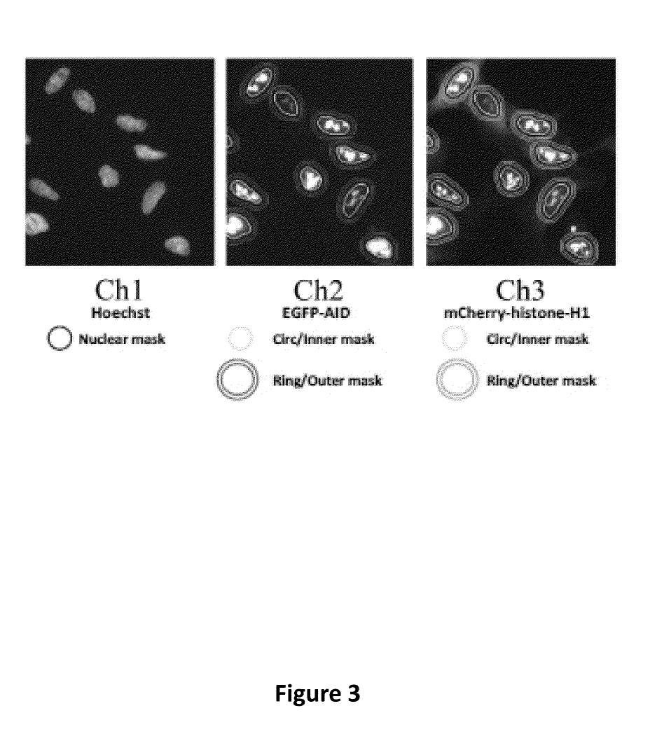

Assays are performed using ArrayScan 3.5, ArrayScan VTI (AS-VTI), and ImageXpress Ultra (IXU) HCS automated imaging platforms. Image acquisition requires input on: the objective; the number of channels to be acquired; the excitation and emission filters; focal offsets required relative to the autofocus point; exposure times; and the number of image fields that need to be captured (Nickischer et al., 2006, Methods Enzymol, 414: 389-418; Williams et al., 2006, Methods Enzymol, 414: 364-89; Dudgeon et al., 2010, Drug Dev Technol., 8: 437-458). Images are acquired in the blue and green fluorescent channels for the primary screen and the blue, green and red channels for the counter screens subsequent to DMSO or LMB treatment. The molecular translocation (MT) image analysis algorithm is utilized to analyze the images acquired on the AS-VTI and to quantify the subcellular distribution and N/C ratios of AID-EGFP and mCherry-H1. On the IXU platform the translocation enhanced (TE) image analysis module is used to analyze the images and extract quantitative fluorescence within a software selectable width (FIG. 3).

Both the MT and TE image analysis algorithms use the Hoechst stained nuclei in channel 1 (Ch1) to define a nuclear translocation mask. Hoechst stained objects in Ch1 that have fluorescent intensities above background with appropriate morphological characteristics (width, length & area) are identified and classified by the image segmentation as nuclei and used to create a nuclear mask and count cells. AID-EGFP images from Ch2 and mCherry-H1 images from Ch3 are segmented into a “Circ/Inner” nuclear region with a mask set by eroding a defined distance (1 pixel) in from the edge of the detected nucleus. AID-EGFP images from Ch2 and mCherry-H1 images from Ch3 are segmented into a “Ring/Outer” cytoplasm region set by expanding out a defined distance away from the edge of the detected.

Quantitation of the digital images includes; mean cell count per image, the average pixel intensity of AID-EGFP (Ch2) or mCherry-H1 (Ch3) in the cytoplasm, the average pixel intensity of AID-EGFP(Ch2) or mCherry-H1 (Ch3) in the nucleus, and a N/C ratio or difference measurement of the average pixel intensity of AID-EGFP (Ch2) or mCherry-H1 (Ch3). The imaging platform, objective and image analysis combination that provides the best data quality, throughput and capacity are selected for all further HCS assay development.

### Example 3

### Establish Additional Shuttling Protein Counter Screens

Counter screens can be developed for the assessment of hits from the primary screen for nonselective effects on nuclear protein import. The counter screens represent multiple modalities of nuclear import mechanisms for the purpose of identifying compounds that selectively inhibit AID nuclear import.

The materials and methods employed in these experiments are now described.

Materials and Methods

Human AID (NM_{—}020661.2) and human histone H1 (NM_{—}005325.3) were purchased from Blue Heron BioTech as wild type cDNAs and subcloned into pcDNA3 and pIRES mammalian expression vectors respectively. The subclones were sequence-verified.

HEK293T cells in 40 μl were plated robotically into 384-well plates using a Multi-Drop (ThermoScientific) such that in 24 hours the cell density is approximately 60% confluency. EGFP-AID and mCherry-H1 plasmids were mixed at a 1:1 molar ratio in Turbofect reagent as per the manufacture's protocol (Fermentas) and dispensed from a trough into the 384-well HEK293T cell cultures with a Perkin Elmer Janus robot liquid handling system. Each well received 25 ng of total plasmid DNA and uniform dispersal of the transfection mixture was ensured by placing the plates on the Perkin Elmer plate mixer at the highest speed setting for 30 sec. Due to the small culture volume, there was no effect on cell attachment to the plates but was critical for uniform transfection. Ranges of both EGFP-AID: mCherry-H1 plasmid ratios and amount of total plasmid DNA required for transfection of cells in 384-well format were evaluated. For current plasmid stocks, a 1:1 plasmid ratio gave the most consistent expression of both proteins. It was found that total plasmid transfected into each well was toxic to cells over 75 ng and did not yield reproducible expression below 25 ng.

Wild type human ACF, hnRNP A1, and TALDO1 cDNAs are obtained from BlueHeron (NM_{—}014576.2, NM_{—}002136.2 and NM_{—}006755.1) cDNAs for ACF and A1 were subcloned into pGEM3 and pIRES, respectively. Criteria used for transient transfection, validating protein expression and determining appropriate N/C ratio baselines are described elsewhere herein. Two counter screens for shuttling proteins, run in parallel enable the identification of AID-selective nuclear import inhibitors. For studies to prove that compounds bind to AID, AID expressed and purified from baculovirus/Sf9 cells are used in biophysical analyses (e.g., surface plasmon resonance and fluorescence polarization anisotropy) to evaluated AID binding affinities for hits of interest.

The results of the experiments are now described.

### Counter Screens

Without being held to any particular theory, AID-selective hits are anticipated to inhibit nuclear import by binding directly to one or more of multiple regions of the NLS in AID and thereby inhibit AID's ability to interact with the machinery that mediates nuclear import. Alternatively, compounds may bind to AID and thereby prevent PKA recognition of AID as a substrate for phosphorylation and thereby enable more rapid nuclear export of AID. This aspect of the assay is to utilize a counter screen to screen out compounds that: (1) non-selectively affect nuclear import and are pleotrophic inhibitors (e.g., nuclear pore binding compounds or GTP-Ran) or (2) that bind to cytoplasmic chaperones that are common to AID and other nuclear proteins (e.g., importin α subtypes). The mechanism(s) of nucleocytoplasmic shuttling of proteins is beginning to be understood and it is believed that nucleoporins, karyopherins and GTP-Ran work together as general protein import factors for some classes of proteins. At the same time these nuclear import chaperones discriminate between individual NLS or families of proteins to facilitate selective and regulated nuclear import. The concurrent H1 counter screen enables selection of compounds that inhibit AID nuclear import but do not affect proteins that are resident in the nucleus (i.e. nuclear imported but nonshuttling) and do not have an effect on general protein expression.

Stable expression for AID-EGFP in HeLa cells has been achieved. For the counter-screening assays, stable expression for co-expressed proteins is also performed. Alternatively, counter screenings using transient co-transfection of proteins are also used. To validate that proteins of interest for counter screening could be co-expressed, Human AID (NM_{—}020661.2) and histone H1 (NM_{—}005325.3) subcloned in the pIRES vector were transiently co-transfected in 6 well plates.

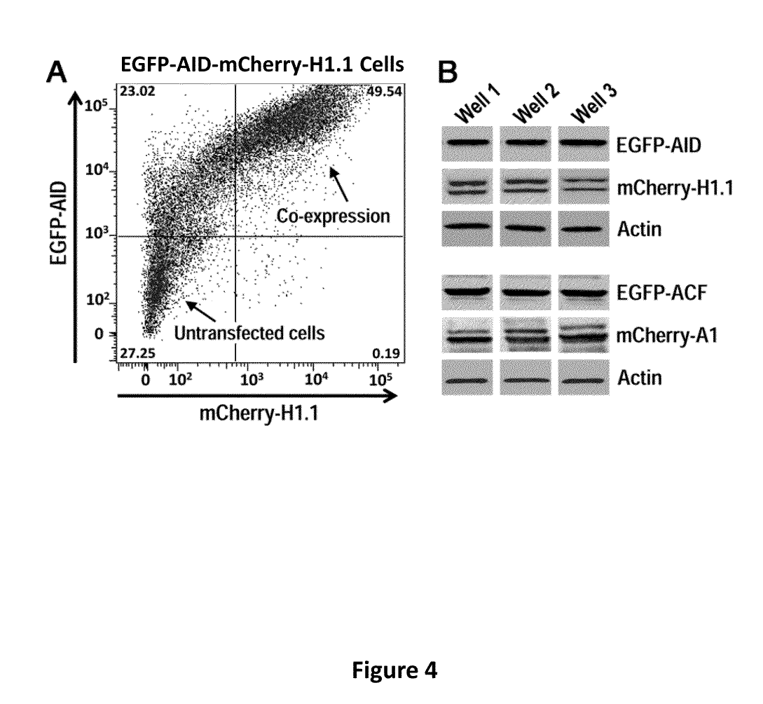

The efficiency of co-transfecting AID-EGFP and mCherry-H1 in 384-well format was evaluated using robotic protocols. The well-to-well coefficient of variance for expressing AID-EGFP and mCherry-H1 was 3% and 5% respectively. The protocol was scaled for transfection of cultures in 6-well cultures to quantify the proportion of cells expressing both proteins. The expression of the recombinant proteins was determined using western blotting. FACS analysis (FIG. 4A) showed that 49.5% of the cells co-expressed both proteins (S.D., 1.9%), 23% only expressed AID-EGFP (S.D., 0.3), 0.2% of the cells only expressed mCherry-H1 (S.D., ND) and 27% of the cells were not transfected by either plasmid (S.D., 1.7%). The overall transfection efficiency was 75% (S.D., +/−10%). Western blots of whole cell extracts from three separately transfected wells revealed full length AID-EGFP (anti-GFP) and mCherry-H1 (anti-HA) (FIG. 4B, upper panel). Similar results were obtained by co-transfecting EGFP-ACF and mCherry-A1 (FIG. 4B, lower panel). Cells transfected in 384-well format were examined by fluorescence microscopy to assess the subcellular distribution of chimeric proteins.

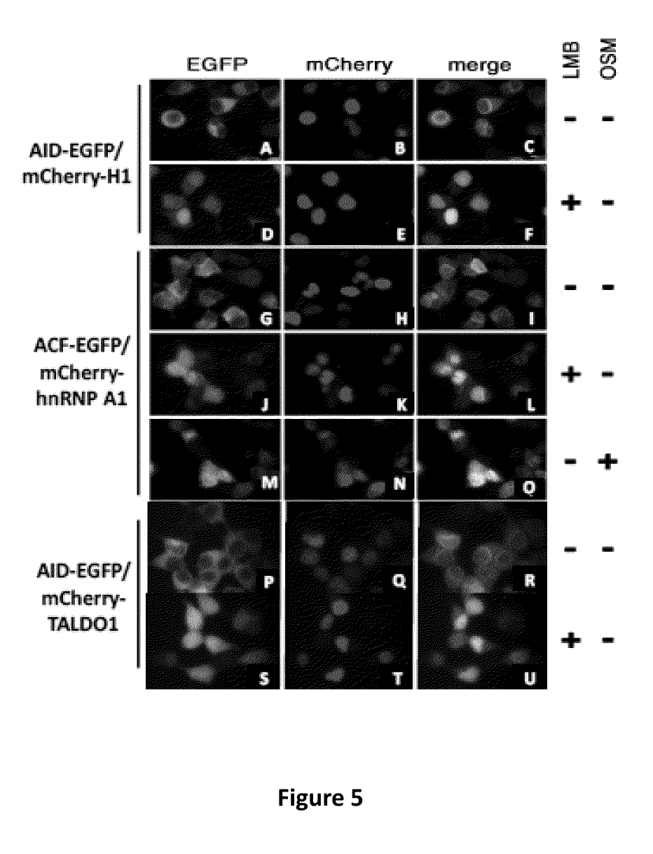

Co-expression of other counter screen proteins and their response to LMB was evaluated using co-transfected HEK293T cells. FIG. 5A through FIG. 5C show an example of live 293T cells in a well from a 384-well plate, 24 hours after transient transfection with AID-EGFP and mCherry-H1 (without FACS) and viewed by epifluorescence microscopy through the excitation and emission filters for Hoechst, EGFP and mCherry along with a composite image. AID-EGFP was localized predominantly in the cytoplasm and most nuclei were dark or very faintly green. mCherry-H1 was nuclear. Comparison of Hoechst stained nuclei to the number of green or red cells showed that >58% were transfected with either AID-EGFP or mCherry-H1, and >64% of the transfected cells expressed both AID-EGFP and mCherry-H1.

Shuttling of AID is only apparent by inhibiting CRM1-dependent nuclear export with LMB and allowing AID to accumulate in the nucleus. LMB is added to cells after treatment with library compounds in order to ‘trap’ AID-EGFP imported into the nucleus during that time and thereby reveal inhibitors of nuclear import. FIG. 5D through FIG. 5F show fluorescent images of cells exposed to 50 ng/ml LMB (Sigma) for 90 min. Cells were tolerant of the methanol solvent used for LMB (e.g., 0.5% methanol). Increasing the amount of LMB in the treatment above 50 ng/ml did not increase the amount of EGFP-AID retained in the nucleus in the transient system, as described above, differed from what is observed for the stable expression system.

Comparing FIG. 5A to 5D, all cells that expressed AID-EGFP exhibited increased retention in their nuclei in response to LMB. All cells expressing AID-EGFP were responsive to LMB but clearly, the amount of AID-EGFP retained in the nucleus was heterogeneous in that some cells exhibit homogeneous cellular fluorescence while others have brightly fluorescent nuclei. The second feature of the assay is that histone H1 fluorescence remained nuclear independently of LMB treatment.

The data presented herein demonstrates that: (1) AID-EGFP and mCherry-H1 can be readily co-expressed as a counter screen assay, (2) LMB conditions that induce nuclear retention of AID do not affect H1 nuclear localization and (3) transient cotransfection could be used in the establishment of either primary or counter screening assay as an alternative to stable co-expression.

Two different counter screens for shuttling proteins are developed for the purpose of running them simultaneously on all confirmed concentration-dependent hits from the primary screen. Wild type human ACF, hnRNP A1 and TALDO1 cDNAs obtained from BlueHeron (NM_{—}014576.2, NM_{—}002136.2, NM_{—}006755.1) were cloned into pGEM3 or pIRES. The cDNAs are subcloned in lentiviral expression vectors from AddGene (pLVTHM using EF1-α promoter) or Genecoepia (pReceiver using the CMV promoter) and transduced into HeLa cells to establish stable co-expression. FACS is used to select cells co-expressing equivalent EGFP and mCherry signals. Assay optimization in 384-well format is performed as described previously.

Data presented herein show images from HEK293T cells 24 hr after being transiently cotransfected with ACF-EGFP and mCherry-hnRNPA1. FIG. 5G through FIG. 5I show images from a 384-well plate for EGFP-ACF, mCherry-hnRNPA1 and two color composite respectively. The bottom panel in FIG. 4B demonstrates expression of full-length proteins based on western blots of cell extracts from three different transfections. ACF and hnRNP A1 are selected because they shuttle using different mechanism than AID and because their function in the cell differs markedly from one another and from AID and H1. ACF binds to RNA through RNP domains that comprise three separate and centrally located RNA Recognition Motifs (RRM), required for ACF's function as an editing site recognition cofactor for APOBEC1 mRNA editing activity in the cell nucleus (Hintersteiner et al., 2010, ACS Chem Biol, 5(10): 967-79). ACF is a shuttling protein with predominantly cytoplasmic cellular distribution as shown in FIG. 5G (Chahine et al., 2009, Pharmacol Rev, 61(3): 358-72; Fukumoto et al., 2011, Cell Structure and Function, 36(1): 57-67; Fornerod et al., 1997, Cell, 90(6): 1051-60). ACF nuclear import is determined by a unique amino acid motif centrally located and a Cterminal, CRM1-dependent nuclear export signal (Chahine et al., 2009, Pharmacol Rev, 61(3): 358-72).

Nuclear retention of ACF is regulated by serine phosphorylation within the N-terminus by PKC (Dong et al., 2009, Nat Struct Mol Biol, 16(5): 558-60; Dudgeon et al., 2010, J. Biomol. Screen. 15: 766-782). Data presented herein show the expected nuclear retention of ACF following LMB treatment (FIG. 5J compared to 5G). HnRNP A1 binds to RNA through two RRMs but unlike ACF, A1 has a predominant nuclear localization (Williams et al., 2006, Methods Enzymol, 414: 364-89). Nuclear import of hnRNP A1 depends on a bipartite NLS motif of basic residues and a short domain known as M9 (Williams et al., 2006, Methods Enzymol, 414: 364-89). HnRNP A1 M9 domain also is required for CRM1-independent nuclear export (Teschendorf et al., 2002, Anticancer Research, 22(6A): 3325-30). FIG. 5K (compared with 5H) shows that hnRNP A1 localization does not respond to LMB. Phosphorylation of residues adjacent to the M9 domain by PKA and PKC are induced during cell stress and lead to hnRNP A1 accumulation in the cytoplasm (as stress granules) (Teschendorf et al., 2002, Anticancer Research, 22(6A): 3325-30; Grassi et al., 2003, Carcinogenesis, 24(10): 1625-35). This makes hnRNP A1 of further interest as a reporter for off-target effects and cell toxicity. Data presented herein show that hnRNP A1 moves into the cytoplasm in osmotically stressed cells (OSM, sorbitol addition to the media) (FIG. 5N).

TALDO1 is broadly expressed in human tissues and participates in nonoxidative pentose phosphate pathway to provide ribose-5-phosphate for nucleic acid synthesis and NADPH for lipid biosynthesis. It is similar to AID in terms of molecular mass, uses the same nuclear importin α proteins as AID (Fukumoto et al., 2011, Cell Structure and Function, 36(1): 57-67), but differs in AID in that TALDO1 has a predominantly nuclear localization (FIG. 5Q).

Although TALDO1 has leucine-rich stretches of amino acids in its C-terminus, shuttling of TALDO1 and CRM1-dependent nuclear export have not been evaluated. It will be determined if LMB induces further nuclear retention of mCherry-TALDO1 but its significance for the present counter screen is that co-expression of this protein with AID enables the selection AID-selective nuclear import inhibitors. Compounds that specifically bind to and inhibit nuclear import of AID and do not alter the subcellular distribution of TALDO 1 (+/−LMB) are prioritized.

TALDO1 in pIRES was transiently co-transfected with AID-EGFP in 293T cells and fluorescence images taken 24 hours post-transfection. AID-EGFP was predominantly cytoplasmic while mCherry-TALDO1 was predominantly nuclear (FIG. 5P through FIG. 5R). These data suggest that at equilibrium, AID and TALDO1 shuttling rates are different and that either TALDO1 is more efficiently imported and retained in the nucleus compared with AID or that TALDO1 nuclear export is slower than that of AID. LMB treatment induces significant nuclear retention of AID-EGFP and colocalization with mCherry TALDO1 but did not noticeably affect the nuclear mCherry fluorescence due to TALDO1 alone (FIG. 5R through FIG. 5U). It is concluded that co-expression of AID and TALDO1 is achievable and that the proteins have different N/C ratios even though they use the same importins.

While transient transfection system is described herein, there are alternatives for creating stable cell lines. Longer-term expression of recombinant proteins has been shown to diminish due to silencing of the CMV promoter (Teschendorf et al., 2002, Anticancer Research, 22(6A): 3325-30; Grassi et al., 2003, Carcinogenesis, 24(10): 1625-35). Therefore, stable cell lines for EGFP-AID (wild type and catalytic mutant) can be created using mammalian expression vectors wherein transcription is driven by the EF1 alpha 1 or integrin beta 1 promoters available from Addgene (Cambridge, Mass.). Further, stable cell lines can be created using the lentivirus gene delivery system from Genecopoia (Rockville, Md.). To address the possibility that long-term expression of AID might be genotoxic, stable expression of EGFP-AID using the Clontech (Mountain View, Calif.) Tet-regulated promoter system (Qin et al., 2010, PloS one, 5(5): e10611) (also available in the lentiviral systems) can be explored. In this system EGFP-AID expression in stable cell lines is suppressed by doxycycline in the media. Removal of doxycycline prior to screening would allow expression of EGFP-AID to appropriate levels before library compounds are added.

It is anticipated that ACF/hnRNP A1 and AID/TALDO1 counter screening assays will meet the criteria required for HCS assays with acceptable z′ factor and high signal-to-noise and signal to background ratios necessary for statistically sound evaluation of compounds that only affect AID nuclear import. AID-EGFP/mCherry-H1 assay is selected as a primary screen as the proteins have markedly different nuclear import mechanisms and the data presented herein indicates a highly functional primary HCS assay. The AID-EGFP/mCherry-TALDO1 may also be used as a primary screen. These counter screens are not inclusive of all potential mechanisms for nuclear import, but, without being held to any particular theory, it is believed that ACF, hnRNP A1, TALDO1 and Histone H1 enable the ability to sample a significant number of protein shuttling mechanisms to be confident in selecting hits as being AID-selective.

All of the studies described herein were performed with live cells stained with Hoechst 33342. However, given the number of plates required to screen a large library, fixation of the cells can be used to ensure that the time frame considered for nuclear import is equivalent for all treatment groups. Fixatives and fixation conditions are optimized to produce images comparable to those of live cells with Hoechst 33342 using established methods (Williams et al., 2006, Methods Enzymol, 414: 364-89; Teschendorf et al., 2002, Anticancer Research, 22(6A): 3325-30; Grassi et al., 2003, Carcinogenesis, 24(10): 1625-35; Johnston et al., 2008, Assay Drug Dev Technol, 6: 505-518; Shun et al., 2011, J Biomol Screen., 16: 1-14; Johnston et al., 2007, Assay Drug Dev Technol., 5: 319-332). It is predicted that the assay is reproducible with an assay signal window that allows for the identification of compounds that inhibit AID nuclear import. Specifically, the highest N/C ratio for AID-EGFP is represented by LMB treatment control wells that are used as the HCS assay's maximum N/C ratio plate controls for normal (uninhibited) nuclear import. The N/C ratio for AID-EGFP in wells treated with DMSO (0.2%) plus MeOH (0.5%) exhibit the lowest N/C ratio for AID-EGFP and are used as the HCS assay's minimum N/C ratio plate controls for fully inhibited nuclear import. Maximizing the gap between these two thresholds and minimizing the well-to-well variance (collectively used in the calculation of the assay's Z′-factor) is sought in assay development. It is anticipated that the assay achieves a Z′-factor ≧0.5 and has a high signal-to-noise and signal-to-background. Compounds that inhibit AID-EGFP nuclear import (N/C ratio) by ≧3 standard deviations below the mean of the LMB-treated control plate N/C ratio (Z-score ≦−3) are flagged as hits.

The possibility that the library may contain compounds that: (i) inhibit CRM1-dependent nuclear export of AID, (ii) enhance nuclear import of AID or (iii) enhance AID nuclear export, cannot be ruled out. Compounds affecting the first two pathways would lead to high N/C ratios in the absence of LMB and will be evident through subsequent testing of hits in qHTS in wells of cells minus LMB treatment. Detecting export activators has a low probability due to the dominant role of the CRM1 export mechanism. It may depend on whether the compound's target/mechanism of action is upstream of downstream of the CRM1 mechanism of action and their potency as enhancers of AID nuclear export relative to retention induced by LMB. Such compounds may bind directly to AID to enhance CRM1-dependent interactions or they may act as activators of the CRM1 pathway. In the latter case they also will be apparent in the ACF/hnRNP A1 counter screen as reduced ACF N/C ratio and may be overcome using higher concentrations of LMB. These compounds are not pursued because although AID nuclear export enhancers will reduce nuclear abundance of AID, they may still allow AID access to the genome which may be sufficient over time to induce SHM and CSR.

### Example 4

### Conduct Validation HCS

The choices made for automating cell plating, compound treatment, and sample preparation have a significant impact on the biology and the consistency of HCS assays (Dudgeon et al., 2010, J. Biomol. Screen., 15: 766-782; Trask et al., 2006, Methods Enzymol, 414: 419-39; Trask et al., 2009, Methods Mol Biol, 2009. 565: 159-86). The AID-EGFP nuclear distribution HCS assay is adapted for automation and to validate its performance during the screening of compounds for a test library (Shun et al., 2011, J Biomol Screen., 16: 1-14). The statistical variability of the assay is estimated in a 3-day assay for signal window, z′ factor determination test (Shun et al., 2011, J Biomol Screen., 16: 1-14). To determine how the assay plate controls perform during the automated process, three independent 5-plate DMSO runs are conducted (Shun et al., 2011, J Biomol Screen., 16: 1-14). To determine how the assay performs in the presence of compounds, the 1,280 compound LOPAC (Sigma) and 446 compound NIH Clinical Collections libraries are screened. However, it is envisioned than any library can be used in the HCS assays of the invention.

The seeding of the EGFP-AID/mCherry-H1 cells into assay plates is automated using the Zoom dispenser (Titertek) or the MicroFlo (BioTek) as described elsewhere (Dudgeon et al., 2010, J. Biomol. Screen, 15: 766-782; Dudgeon et al., 2010, Drug Dev Technol., 8: 437-458; Trask et al., 2006, Methods Enzymol, 414: 419-39; Trask et al., 2009, Methods Mol Biol, 2009. 565: 159-86). Plate controls to define the assay signal window and for quality control review are placed in the wells in columns 1, 2, 23 and 24 of the 384-well assay plate. Compounds are added to the 320 wells in columns 3-22, providing 32 max control wells (LMB) and 32 min control wells (DMSO) that are evenly distributed among the wells of columns 1, 2, 23 and 24. Compound and control additions are automated on the Bravo (Velocity 11), Evolution P3 (Perkin Elmer) or Janus (Perkin Elmer) automated liquid handling platforms outfitted with 384-well transfer heads. After the required compound exposure and LMB post-incubation times, the cells are subjected to automated fixation, Hoechst 33342 staining and PBS washing of the cell monolayers as described elsewhere (Dudgeon et al., 2010, J. Biomol. Screen, 15: 766-782; Dudgeon et al., 2010, Drug Dev Technol., 8: 437-458).

After an initial screen of a small collection of compounds for identification of the time-dependent effects of active compounds, cytotoxic compounds and fluorescence interference compounds, the optimal length of compound exposure is selected for a larger screen. On the second day of operations, this time point is used to repeat validation screens. Compounds identified as active in both runs are confirmed in 10-point IC50 dose-response assays Images from concentration-dependent hits are evaluated for the effectiveness of compounds in functional endpoint assay and to detect signs of cytotoxicity by morphology or reduced cell adherence. Compounds also are evaluated for auto-fluorescence and effects on overall AID-EGFP fluorescence due to changes in its expression by western blotting. While not wishing to be bound by any particular theory, it is anticipated that small molecules that can inhibit AID-EGFP nuclear import have an effect within 1-2 hours. However, initial testing is performed on the first day of operations with compound addition to live cultures for 1, 3 and 24 hours prior to LMB treatment, fixation and imaging.

The data presented herein shows a screen of the 1,280 compound LOPAC library in 4×384-well plates (FIG. 6). As expected, cells (2,500 cells/well) treated with DMSO alone had predominantly cytoplasmic AID (set a 100%) whereas those treated with solvent and LMB alone had predominantly nuclear AID (set at 0%). The LOPAC screen was performed using 20 μM of each compound in duplicate with a 3-hour incubation followed by LMB treatment. The assay had a Z′ factor of 0.78 and R²=0.9. Using a hit cut off of 50% inhibition of AID-EGFP nuclear import, 5 compounds out of 1,280 were considered hits. Three of these hits were false positive autofluorescent compounds (example M6545), leaving two potential hits (a hit rate for the pilot screen=0.2%). These hits (SID 53777289 and SID 537782) are known to be NFkB and VEGFR tyrosine kinase inhibitors, respectively. Further optimization of the assay includes optimizing day-to-day and plate-to-plate consistency as well as determining whether a lower threshold (e.g. 35% inhibition) would identify additional compounds. SID 53777289 is an encouraging finding as a prior screen that this compound also inhibited nuclear import of the glucocorticoid receptor (Johnston et al., 2012, Assay Drug Dev Technol, 10(5): 432-456) and as described above, NFkB has been implicated in AID regulation. Thus, identification of a known nuclear import inhibitor demonstrates the ability of the present assay to correctly identify the subset of compounds, within a large set of compounds, that act as nuclear import inhibitors.

For the baseline N/C ratios, the statistical variability of the assay is estimated in a 3-day test assay for signal window and Z′-factor coefficient determination. The assay signal window is measured and the Z score of the AID-EGFP nuclear distribution HCS assay is determined by capturing data from two full plates each of max (LMB treated) and min (DMSO) controls tested in three independent experiments. The % CVs of max and min controls, the signal to background ratios, and the intra-plate, inter-plate, and day to day reproducibility and variability of the assay signal window are determined. These data allow the establishment of quality control, statistical parameters, and the selection of an appropriate active criterion (e.g. ≧50% inhibition as the initial final cutoff) for the HCS campaign (Shun et al., 2011, J Biomol Screen., 16: 1-14). To determine how the assay plate controls perform during the automated process, 3 independent 5-plate DMSO validation experiments are conducted to mimic three days of automated screening operations, the sole difference being that DMSO is added to wells instead of compounds (Shun et al., 2011, J Biomol Screen., 16: 1-14). The 3×5-plate DMSO validation data is reviewed to confirm that the plate controls continue to provide a robust and reproducible signal window with adequate z′ factors and signal to noise ratios to estimate the rate of false positives. A normality evaluation and analysis of variance (ANOVA) is performed to identify any significant row/column effects or other positional biases.

The z′ factor plate-based statistical scoring method is utilized to identify compounds that behave as outliers compared to the other substances (n=320, no controls) tested on the assay plate for selected HCS multiparameter measurements output by the image analysis module (Dudgeon et al., 2010, J. Biomol. Screen, 15: 766-782; Dudgeon et al., 2010, Drug Dev Technol., 8: 437-458). For example, the selected object counts per valid field of view (SCCPVF) parameter derived from the Hoechst DNA staining in channel 1 is utilized to identify compounds that were acutely cytotoxic. A deviation of 4σ below the sample average selected object counts per field of view on the plate (SCCPVF Z scores <−4) is designated as the threshold or cut-off for cytotoxic compounds. Z-scores for two fluorescent intensity parameters from each of the three fluorescent channels are calculated: the mean nuclear total intensity and the mean nuclear average intensity from the Hoechst channel (Ch1); the mean Circ/Inner (nuclear) average intensity and the mean Ring/Outer (cytoplasm) average intensity from the EGFP channel (Ch2); and the mean Circ/Inner (nuclear) average intensity and the mean Ring/Outer (cytoplasm) average intensity from the mCherry channel (Ch3). Compounds that exhibited a deviation of 4σ above (Z-scores >4) or below (Z-scores <−4) the sample average in either one or both of the intensity parameters from each channel is considered fluorescent outliers.

As hits are identified and validated through the counter screens, cross target queries of the PubChem and University of Pittsburgh Drug Discovery Institute (UPDDI) databases are performed to identify and eliminate hit compounds that have promiscuous bioactivity profiles (Johnston et al., 2008, Assay Drug Dev Technol, 6: 505-518; Johnston et al., 2007, Assay Drug Dev Technol., 5: 319-332; Johnston et al., 2007, Assay and Drug Development Technologies, 5(6): 737-750; Johnston et al., 2009, Assays and Drug Development Technologies, 7: 250-265; Soares et al., 2010, Assay Drug Dev Technol., 8: 152-174). It is determined how many bioassays each of these compounds have been screened against and the number of active and confirmed active (concentration dependent) flags in the databases. Both the number and nature of the targets they have been confirmed against are considered to evaluate the selectivity and specificity of the hits that alter EGFP-AID nuclear localization. Promiscuous non-selective hits are eliminated from further consideration. Leadscope Enterprise 2.4.6-1 software is used to cluster and classify the chemical structures of the confirmed hits that alter AID-EGFP nuclear localization by recursive partitioning (Johnston et al., 2007, Assay Drug Dev Technol., 5: 319-332; Johnston et al., 2007, Assay and Drug Development Technologies, 5(6): 737-750; Johnston et al., 2009, Assays and Drug Development Technologies, 7: 250-265; Soares et al., 2010, Assay Drug Dev Technol., 8: 152-174; Johnston, 2008, Automated High Content Screening Microscopy, in High Content Screening, 25-42). To ensure that the structural classes represented in the confirmed hits are enriched relative to the primary HCS library, similar searches using Leadscope are performed. QikProp (Schroedinger, Inc.), Leadscope Enterprise 2.4.6-1, and ADMET Predictor v. 3.0.0 (SimulationsPlus) are also employed to predict the physicochemical properties of hits and compounds that are predicted to have the best bioavailability are selected before they are evaluated in the functional endpoint assays.

The primary assay and subsequence counter screens is sufficiently robust for the identification of nuclear import inhibitors from which AID-selective molecular probes are identified. Hits are further selected for those that selectively inhibit AID nuclear import in a dose-dependent manner as determined by a 10-point IC50 dose-response evaluation of each hit, and similarly triaged for cytotoxic compounds as described below. Given a 0.2% hit rate, it is anticipated that <400 initial hits will result from the screen of a 220,000 compound UPDDI library.

### Example 5

### Develop and Optimize CSR and SHM Secondary Endpoint Assays

AID nuclear import and deaminase activity on genomic DNA are required for both SHM and CSR, while shuttling and the CRM1 binding domain of AID only are required for CSR. Both these functionalities have been linked to oncogenic mutations and translocations. The ultimate proof-of-concept for AID nuclear import inhibitors is that they inhibit SHM and CSR, and these assays are more practical for vetting compounds than sequencing the immunoglobulin gene locus for AID-dependent point mutations. These assays become more practical for vetting compounds once a small number of relevant hits are identified.

The SHM and CSR assays are well established in the field, only requiring optimization to adapt them for a moderate to large number of compounds for functional endpoint analysis. These assays may require minor adaptations for parallel analysis of a potentially large number of future candidate hits, and their robustness with regard to application for broad chemical screenings (e.g. tolerance for DMSO and general cytotoxicity) can be assessed. The adaptability of these assays for use in parallel screening approaches (in 96-well plates) is evaluated and their tolerance to DMSO is tested.

### Cell Viability

Cell viability in a dose-response analysis is determined by flow cytometry using Promega LIVE/DEAD stain included in all of the functional endpoint evaluations. An alternative system utilizes exogenous AID-dependent onset of resistance to the tyrosine kinase inhibitor Imatinib in the BCR-ABL-expressing K562 human CML cell line as a read-out of mutation activity (Orthwein et al., 2010, J Exp Med, 207(12): 2751-65).

### Class Switch Recombination Assays

Candidate compounds are first screened using the cell line CH12F3. Those successfully reducing CSR in this assay are then confirmed in mouse primary B cells.

CH12F3 Cell Line Assay:

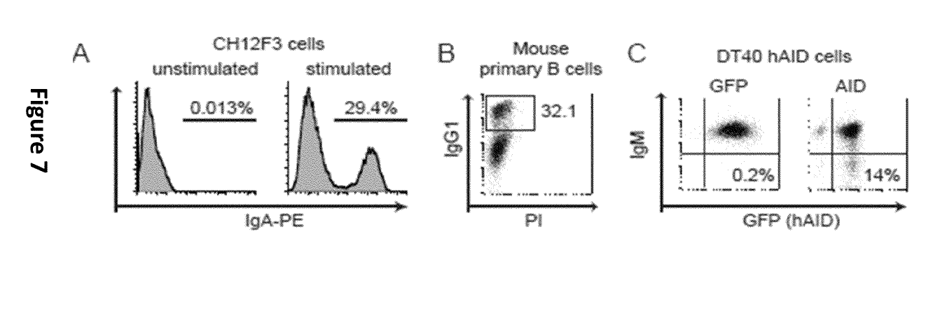

IgM+ mouse lymphoma CH12F3-2 cells efficiently undergo IgM to IgA CSR in vitro (Nakamura et al., 1996, International Immunology, 8(2): 193-201). Stimulation with 1 ng/ml TGFβ1, 10 ng/ml recombinant murine IL-4 and 1 μg/ml functional grade purified anti-mouse CD40 for 3-4 days typically results in ˜30-50% IgM to IgA switching measured by flow cytometry using anti IgA-PE (FIG. 7A). The CH12F3 cell assay has previously been validated while demonstrating the action of indirect AID inhibitors on CSR. To different extents, both HSP90 inhibitors as well as shRNA-reduced expression of the AID cofactor DnaJa1 resulted in linear dose-dependent inhibition of CSR (Orthwein et al., 2010, J Exp Med, 207(12): 2751-2765; Orthwein et al., 2012, The EMBO Journal, 31(3): 679-691). Thus, CH12F3 are suitable to test the ability of the candidate compounds to affect a critical AID endpoint. However, since it is estimated that <20 compounds to get to this stage, and the need for dose-dependent analysis, the assays are optimized for 24-well plates. One million CH12F3 cells are plated and stimulated as above in the presence of the compounds at 6 different concentrations in duplicate and analyzed by flow cytometry after 3 days. Based on previous results (Orthwein et al., 2010, J Exp Med, 207(12): 2751-2765; Orthwein et al., 2012, The EMBO Journal, 31(3): 679-691), a >15% decrease in CSR is considered significant.

Primary B Cells:

Resting mouse primary B cells are purified by CD43-depletion (using MACS, Miltenyi Biotech) from total splenic lymphocytes. This typically yields 20×10⁶ resting B cells/spleen. Cells are loaded with CFSE to monitor proliferation and 10⁶ cells/well are seeded in 24-well plates with 5 μg/ml LPS and 5 ng/ml mouse IL-4. About 25 to 40% of the cells switch to IgG1 by day 4 post-stimulation (FIG. 7B). The candidate compounds are added 24 h post-activation to allow time for the cells to enter the cell cycle and induce AID. Compounds are tested at 2-6 different concentrations in triplicate (according to CH12F3 and preliminary toxicity results). Three days after adding the compounds, CSR to IgG1 is measured by flow cytometry using anti-IgG1-APC excluding dead cells by gating out propidium iodide+ cells. In this case, CFSE allows the discrimination of any toxic effects from actual inhibition of CSR. CSR is analyzed in cell populations that have undergone the same number of cell divisions; allowing the detection of effects on CSR even for compounds that are toxic and slow down cell proliferation. CSR is evaluated over a 7 point concentration range for each compound and the dosing interval over three days is assessed from twice daily to a single initial dose.

Alternatively, to test AID activity in class switching, primary IgM+ B cells from mouse spleen are stimulated in vitro with anti-CD40 and IL-4, which induces proliferation and Ig class switching. Alternatively, primary IgM+ B cells from mouse spleen are stimulated in vitro with anti-IgM, which only induces proliferation. Generation of IgG+ cells are measured at day 5 of stimulation by flow cytometry and by measuring IgG secretion in supernatants by ELISA. The assay is quantitative because AID mutagenic activity is rate limiting for CSR (Ichikawa et al., 2006, J Immunol, 177(1): 355-61). The CSR assay has already been adapted to 96-well plates, so it is amenable to large-scale parallel screening.

### Somatic Hypermutation Analysis

Since AID initiates both CSR and SHM in the same way, it is expected that any inhibition of CSR will translate into SHM inhibition. There is no short-term in vitro assay for SHM, as mutations require time to accumulate to a detectable extent by any phenotypic assay. There is also no human or mouse B cell system for this. Therefore, SHM is monitored using the chicken B cell line DT40, which has been modified to prevent Ig gene conversion and therefore diversifies the IgVL region by SHM (Arkawa et al., 2002, Science, 295(5558): 1301-1306; Arkawa et al., 2004, PLoS Biology, 2(7): e179). An AID−/− sub-line of DT40 that has been complemented with human AID-IRES-GFP (FIG. 7C) is utilized for this assay. It has been previously demonstrated that chicken and human AID shuttle identically in DT40 cells and that all AID shuttling mechanisms are conserved (Honjo et al., 2002, Annu Rev Immunol, 20: 165-196). The read-out of this assay is simple: a proportion of the mutations introduced by AID in the IgVL of this cell line inactivate the Ig gene and can be detected as IgM-loss variants by anti-IgM flow cytometry. This assay requires multiple populations for each compound to account for the stochastic nature of Ig-inactivating mutations. The median proportion of IgM-loss cells appearing over time in multiple populations is proportional to the SHM activity (Orthwein et al., 2010, J Exp Med, 207(12): 2751-2765; Arkawa et al., 2002, Science, 295(5558): 1301-1306). Importantly, inhibiting AID indirectly with HSP90 inhibitors in this assay shows a linear dose-response on SHM, and a 50% decrease was able to be detected; both measured by IgM-loss but also by determining mutation frequency after sequencing IgVL (Orthwein et al., 2010, J Exp Med, 207(12): 2751-2765). It is estimated that using 10 populations with each drug is sufficient to significantly evaluate decreases of >20%. Populations of 50,000 IgM+ cells are grown in 24-well plates in the presence of the compound and the IgM phenotype analyzed by flow cytometry using anti IgM-PE after 3 weeks, splitting daily 1:2 to maintain exponential growth (DT40 doubling time is 10 h). This requires that each compound is evaluated at different doses and dosing intervals so as to not interfere with long term cell growth; which has been demonstrated is an important consideration for HSP90 inhibitors (Orthwein et al., 2012, The EMBO Journal, 31(3): 679-691). Since only a handful of compounds are expected to get to this stage, this tailored optimization for each compound is feasible.

Alternatively, SHM activity is tested using the GFP-stop reversion assay in HEK293 cells (Bachl et al., 1999, European Journal of Immunology, 29: 1383-9), slightly modified as described (Ichikawa et al., 2006, J Immunol, 177(1): 355-61). In this assay, an impaired GFP gene under control of the CMV promoter can be reactivated by AID mutator activity in AID-transfected cells. SHM activity is easily and quantitatively assessed by flow cytometry for GFP+ revertants. Conventionally, this assay is run in 6-well plates, but it is adapted to smaller plate volumes depending on throughput need.

### Example 6

### Validate Compound-Target Interaction

Compounds that inhibit nuclear import of AID and AID functional endpoints and do so in an AID-selective manner, are validated as binding to AID through BIAcore quantitative analyses using immobilized recombinant tagged AID. Recombinant AID the BIAcore T100 is used to conduct surface plasmon resonance measurements. This enables further selection of hits that have the highest probability of success in therapeutic development by identifying compounds with the highest affinity for AID, the highest association constant (kₐ) and lowest dissociation constant (k_{d}). Anti-GST antibody is covalently immobilized onto two flow cells on a BIAcore CMS chip and AID-GST captured on one flow cell and GST to the other cell (as a reference for subtraction). AID-6×His is captured onto a Ni²⁺ charged nitrilotriacetic acid (NTA) sensor chip surface with a blank flow cell used as a reference. Alternatively, an anti-6×His antibody capture method is used. The amount of target captured and temperature of the binding conditions (4° C. to 45° C.) is optimized to give the best binding response with respect to the varying molecular weights of compounds of interest. The typical flow rate for a binding experiment is 30 μL/min for 2-3 min for the association portion of the experiment and 5 min for dissociation in buffer where a ˜20% reduction in response is expected to be seen to get an accurate k_{d}. The amount of ligand bound and the rate at which the compound is flowed over the surface is adjusted to determine either kinetic (kₐ and k_{d}) or affinity (equilibrium disassociation constant K_{D}) constants. The working range of the T100 is 10³ to 10⁷ M-1 s-1 and 10⁻⁵ to 0.5 s-1 for kₐ and k_{d} measurements, respectively. Kinetic data is obtained in a two-fold dilution series over a 10 nM to 100 μM range. The data is analyzed using Evaluation software and Prism4 (GraphPad, Inc) for nonlinear regression analysis of affinity measurements plotting the response at equilibrium (R_{eq}) versus the concentration.

Concurrently, hits are validated as AID nuclear import inhibitors by demonstrating their ability to disrupt AID interaction with importins. Extracts from cells transiently co-transfected with differentially tagged AID and importins α1, α3, or α5 are treated with hit compounds over a range of concentrations before and during the co-IP reactions. Extracts treated with DMSO alone serve as the control for uninhibited co-IP recovery of proteins. Recovery of proteins in the co-IPs are evaluated by PAGE and western blotting and quantified by Phosphorimager scanning densitometry as described recently (Orthwein et al., 2010, J Exp Med, 207(12): 2751-2765; Orthwein et al., 2012, The EMBO Journal, 31(3): 679-691). Importins al, 3 and 5 predicted to interact with AID for nuclear import are quantified for their co-IP with AID in untreated extracts and their yields represent the ‘uninhibited controls’ and serve as a basis for comparison to the yields of these proteins from co-IP of hit-treated extracts. Co-IP are conducted using both the tag on AID and repeated with the tag on the interacting proteins in hit-treated and untreated extracts to validate the findings. Co-IP with other known AID interacting proteins such as CTNNBL1 (Orthwein et al., 2012, The EMBO Journal, 31(3): 679-691), an importing-like protein known to bind to the NLS of splicing factors for nuclear import (Ganesh et al., 2011, The Journal of Biological Chemistry, 286(19): 17091-17102) and Hsp90/Hsp40 (Orthwein et al., 2010, J Exp Med, 207(12): 2751-2765; Orthwein et al., 2012, The EMBO Journal, 31(3): 679-691) are evaluated to determine the breadth of interactions with AID that are affected by nuclear import inhibitors. Importins shown not to be involved in AID nuclear import serve as co-IP controls for nonspecific interactions. Nuclease digestion is used during co-IP to evaluate RNA or DNA bridging.

### Example 7

### AID-EGFP-HeLa HCS Assay LOPAC Screen

The data presented herein describes a high content screen comprising HeLa cells modified to express AID-EGFP. The screen was conducted to identify compounds that inhibited the nuclear import of AID, as described elsewhere herein.

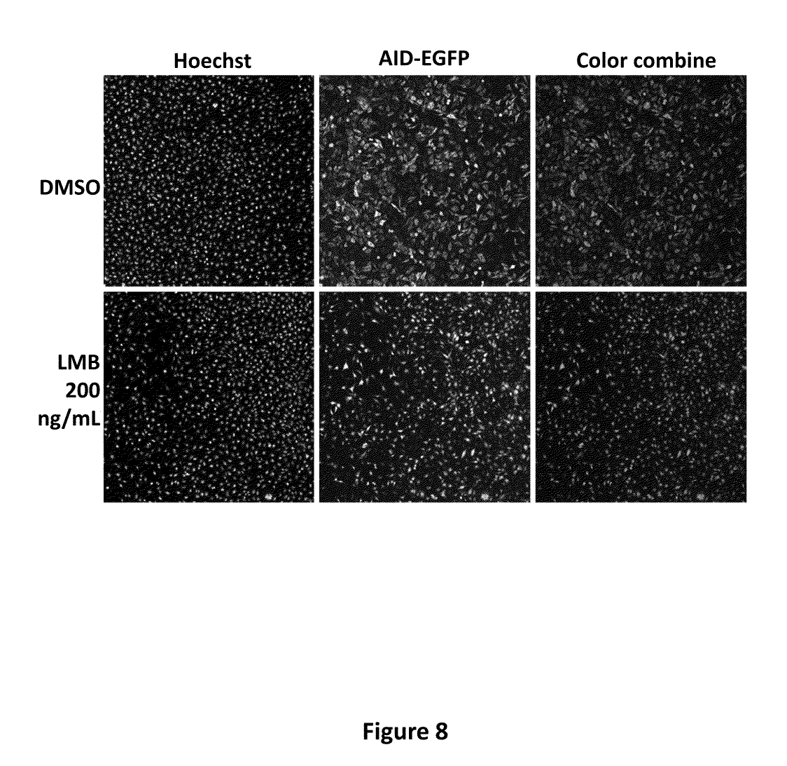

A cell line, AID-EGFP-HeLa cells, were generated which stably express AID-EGFP. HeLa cells were transduced with a lentiviral vector comprising a nucleic acid sequence which encodes AID-EGFP. AID-EGFP-HeLa cells were grown in DMEM/F12 50/50 (Cellgro), supplemented with 10% FBS and Pen/Strep/Fungizon. It is observed that AID-EGFP is predominantly localized in the cytoplasm in untreated, or DMSO treated, AID-EGFP-HeLa cells (FIG. 8). AID-EGFP-HeLa cells have been frozen to prepare a frozen cell bank, and have been expanded from frozen vials in culture.

For the LOPAC high content screen (HCS), 2,500 AID-EGFP-HeLa cells were cultured per well on a 384-well plate. Cells were seeded and cultured overnight. Cells were exposed to 20 μM of test compound for 3 hours, followed by a 200 ng/mL LMB treatment for 3 hours. Experiments were run in duplicate. LMB treatment of control AID-EGFP-HeLa cells (i.e. not treated with test compound) results in approximately 80% nuclear localization (FIG. 8).

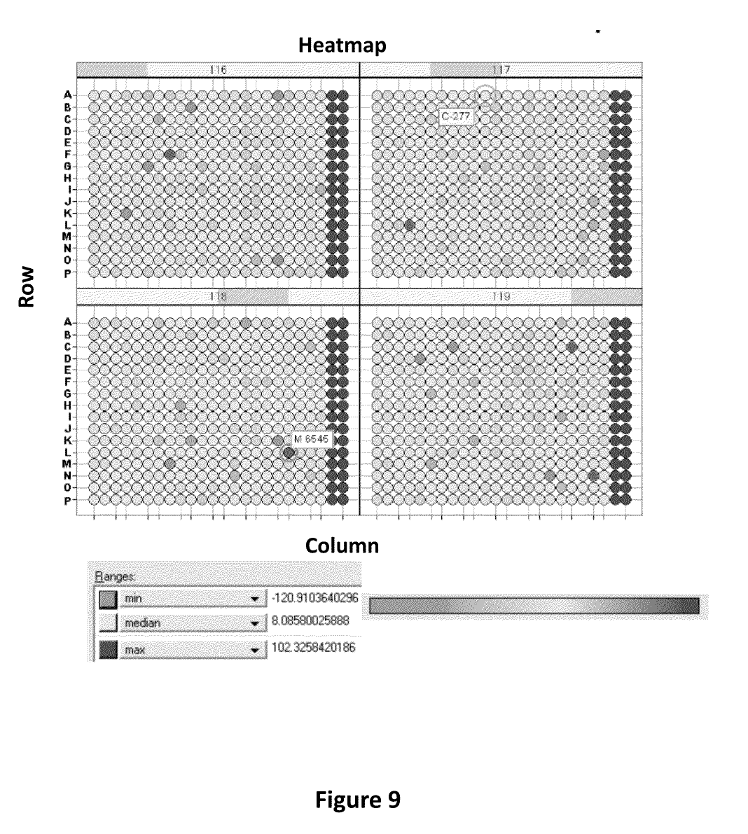

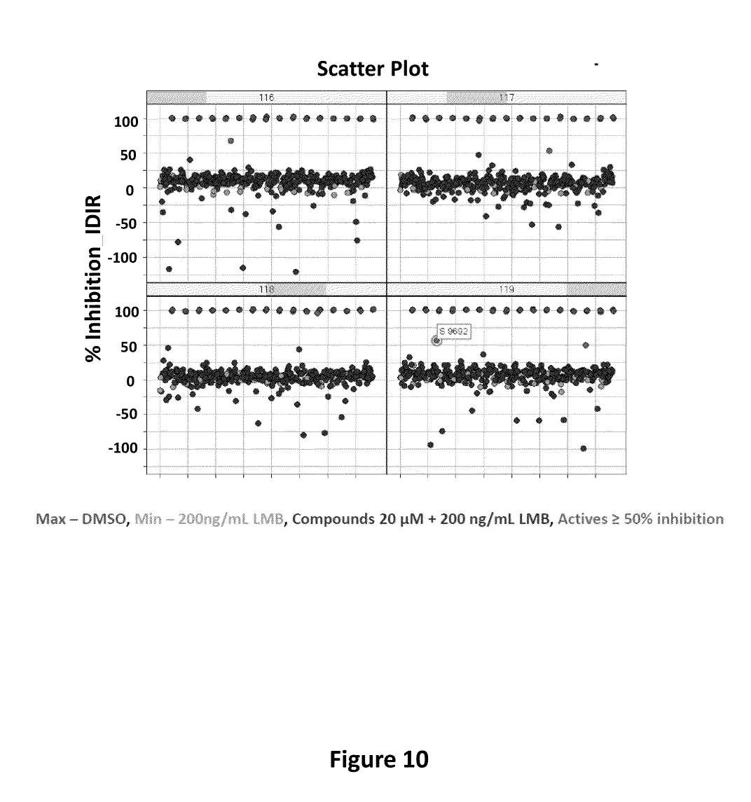

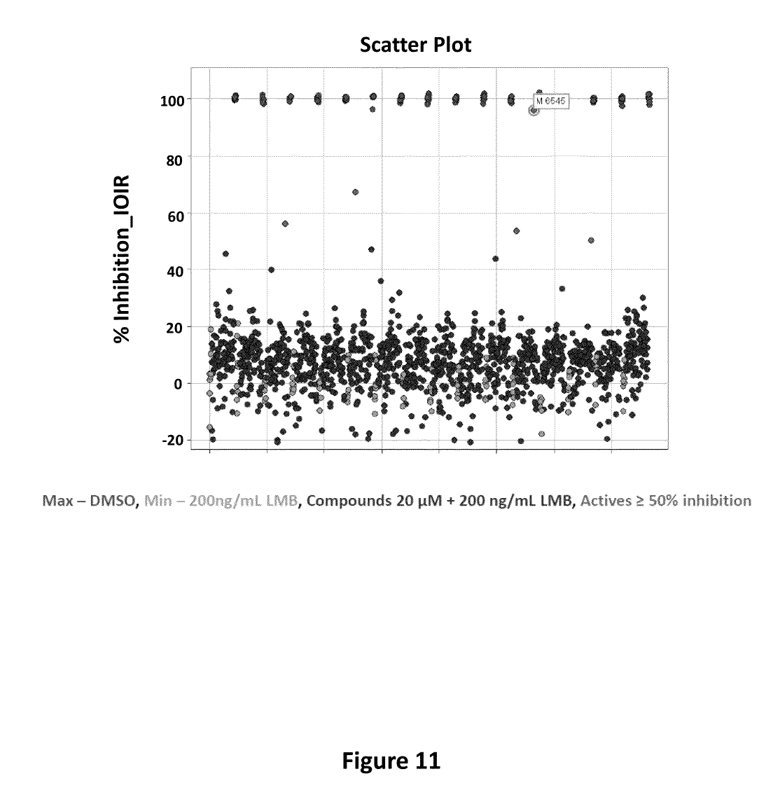

FIG. 9 depicts a heat map of 4 different 384-well plates, while FIG. 10 depicts a scatter plot of the 4 different 384-well plates. Maximum values, set to 100% corresponds to conditions of DMSO treated cells. Minimum values, set approximately to 0%, corresponds to conditions of only 200 ng/mL LMB treatment. Cells subjected to both a test compound and LMB treatment were evaluated for the resulting cellular localization of AID-EGFP, with a greater cytoplasmic localization, compared to LMB only, referring test compounds that inhibit nuclear import. For this screen, active compounds were defined as compounds that resulted in greater than 50% inhibition. FIG. 11 depicts a scatter plot overlay of all 4 plates demonstrating the % inhibition of all test compounds examined Five compounds resulted in inhibition greater than 50%.

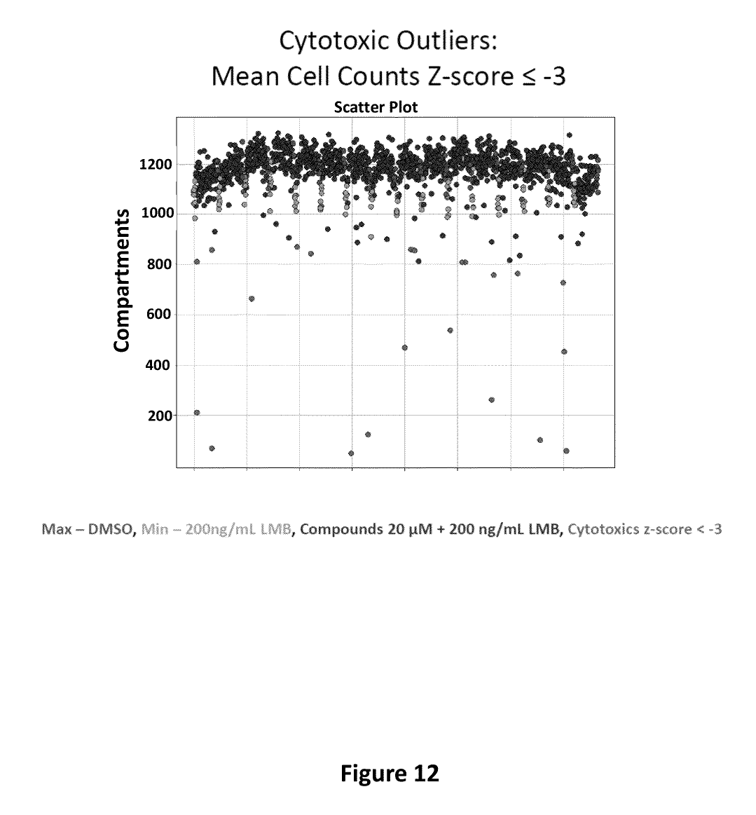

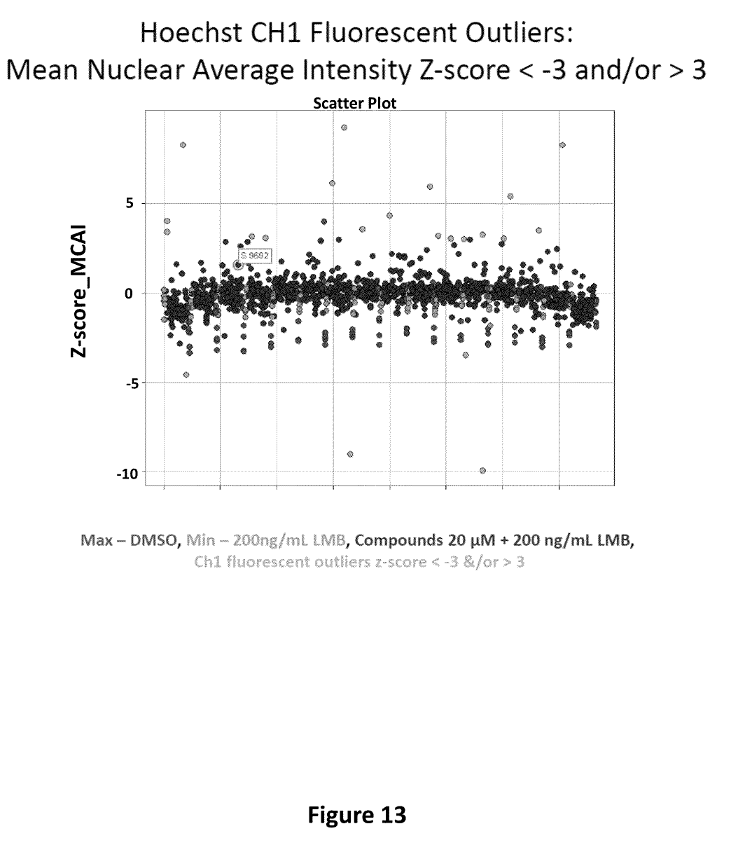

Test compounds were evaluated for being cytotoxic and fluorescent outliers. FIG. 12 depicts a scatter plot demonstrating the analysis of determining whether test compounds are cytotoxic outliers. As depicted, cytotoxic compounds were defined as those compounds that had a mean cell count z-score ≦−3. FIG. 13 depicts a scatter plot demonstrating the determination of whether test compounds are fluorescent outliers, in terms of channel 1 Hoechst fluorescence. As depicted, channel 1 fluoresecent outliers were defined as those compounds resulting in a mean nuclear average intensity z-score ≦−3 or ≧3.

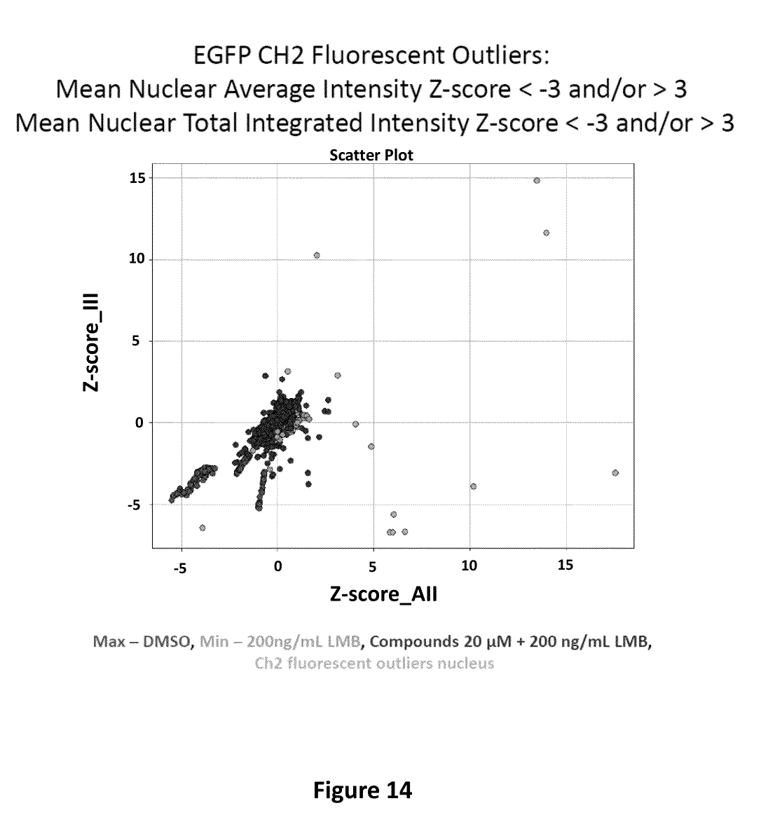

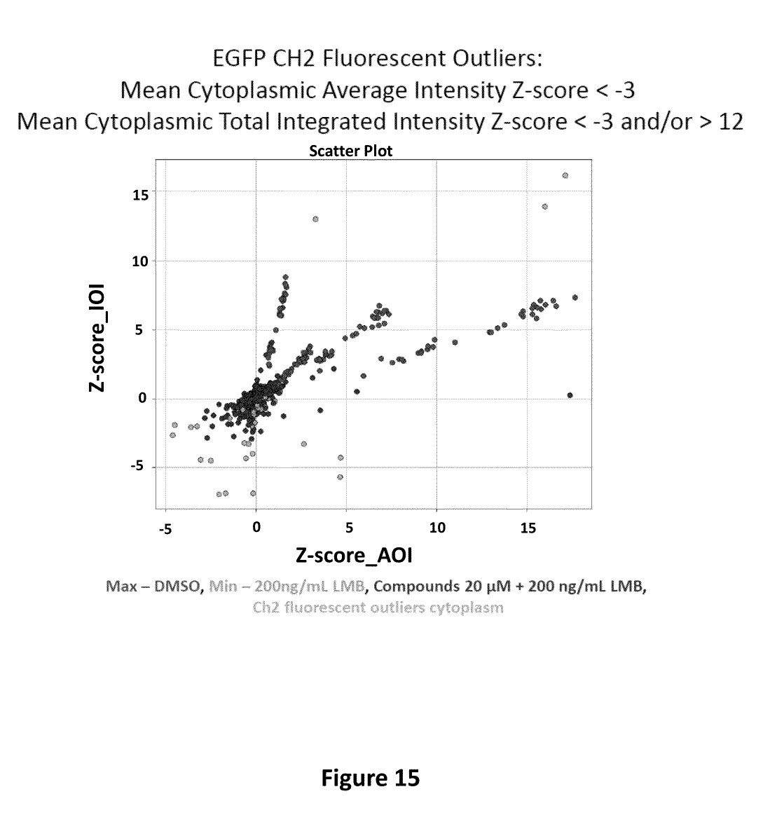

In determining whether test compounds were fluorescent outliers, in terms of channel 2 EGFP fluorescence, outliers of both nuclear and cytoplasmic EGFP fluorescence was examined FIG. 14 depicts a scatter plot demonstrating the determination of whether test compounds are fluorescent outliers, in terms of channel 2 EGFP nuclear fluorescence. As depicted, channel 2 EGFP nuclear fluorescent outliers were defined as those compounds resulting in a mean nuclear average intensity z-score ≦−3 or ≧3 or a mean nuclear total integrated intensity z-score ≦−3 or ≧3. FIG. 15 depicts a scatter plot demonstrating the determination of whether test compounds are fluorescent outliers, in terms of channel 2 EGFP cytoplasmic fluorescence. As depicted, channel 2 EGFP cytoplasmic fluorescent outliers were defined as those compounds resulting in a mean cytoplasmic average intensity z-score ≦−3 or a mean cytoplasmic total integrated intensity z-score ≦−3 or ≧12.

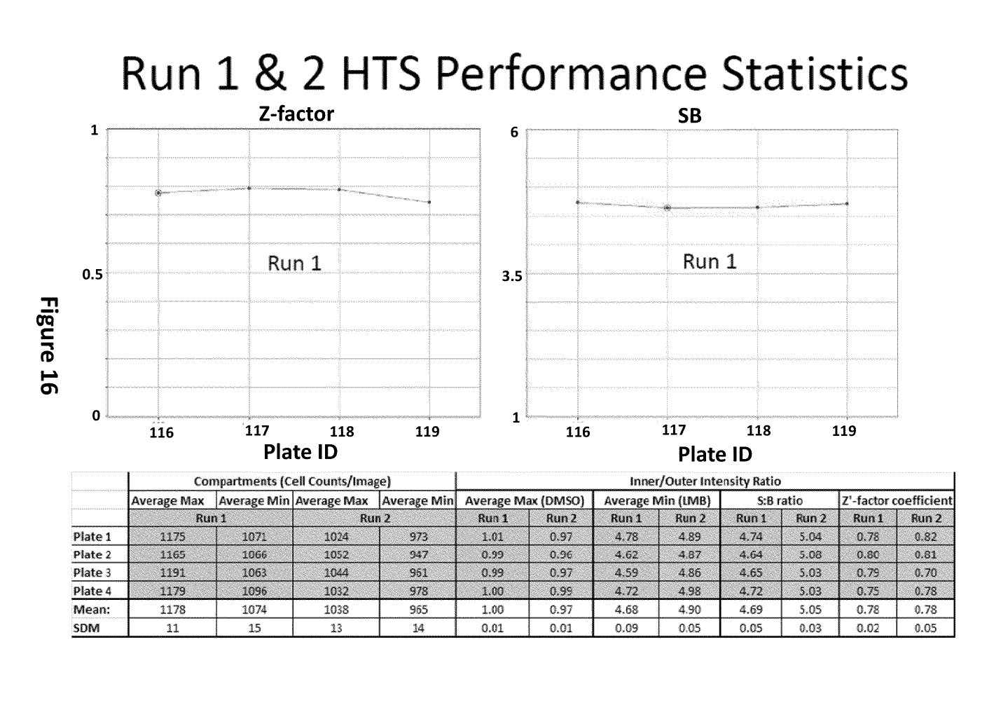

The screening assay was performed twice to examine the consistency of the screen. FIG. 16 depicts the performance statistics of run 1 and run 2, which demonstrates that cell counts, average maximum, average minimum, S:B ratio, and Z′-factor coefficient were similar among Run 1 and Run 2.

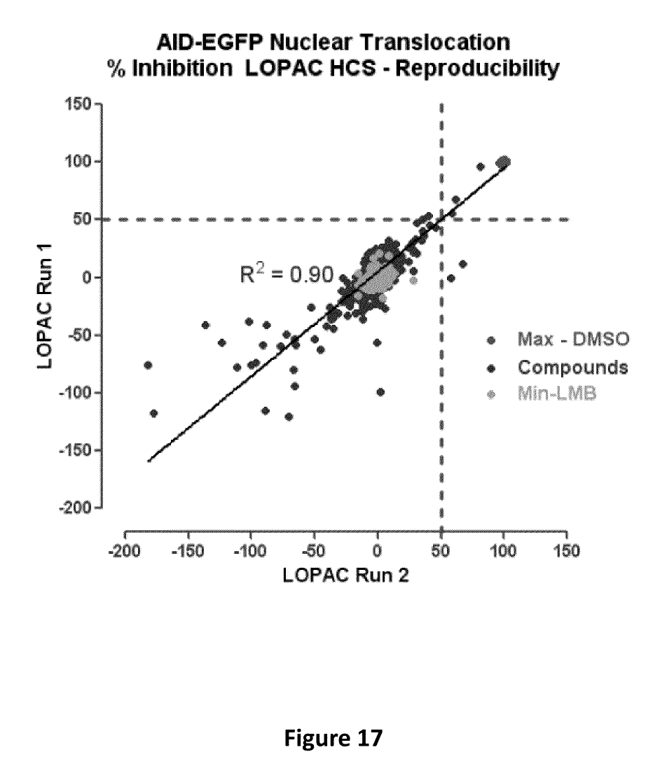

The reproducibility of the percent inhibition of AID-EGFP nuclear translocation, mediated by compounds from the LOPAC screen during two individual runs of the screen (FIG. 17) shows that the screen demonstrates exceptional consistency and reproducibility. The percent inhibition of each compound, along with minimum (LMB) and maximum (DMSO) values, is plotted for each run, as shown in FIG. 17. As depicted therein, a fit of the data from the two runs produces an R2 value of 0.90, thereby demonstrating the reproducibility of the screen.

The LOPAC screen described herein identified five compounds which induced greater than 50% inhibition of AID nuclear import. The percent inhibition of all test compounds is depicted in FIG. 10, which shows the 5 unique compounds that induced percent inhibition above the 50% inhibition threshold. The five identified compounds are Bay 11-7085 (Pubchem SID 53777289), SU 5416 (Pubchem SID 53778216), Ellipticine (Pubchem SID 53777637), Mitoxantrone (Pubchem SID 53777885), and SU 6656 (Pubchem SID 53778085).

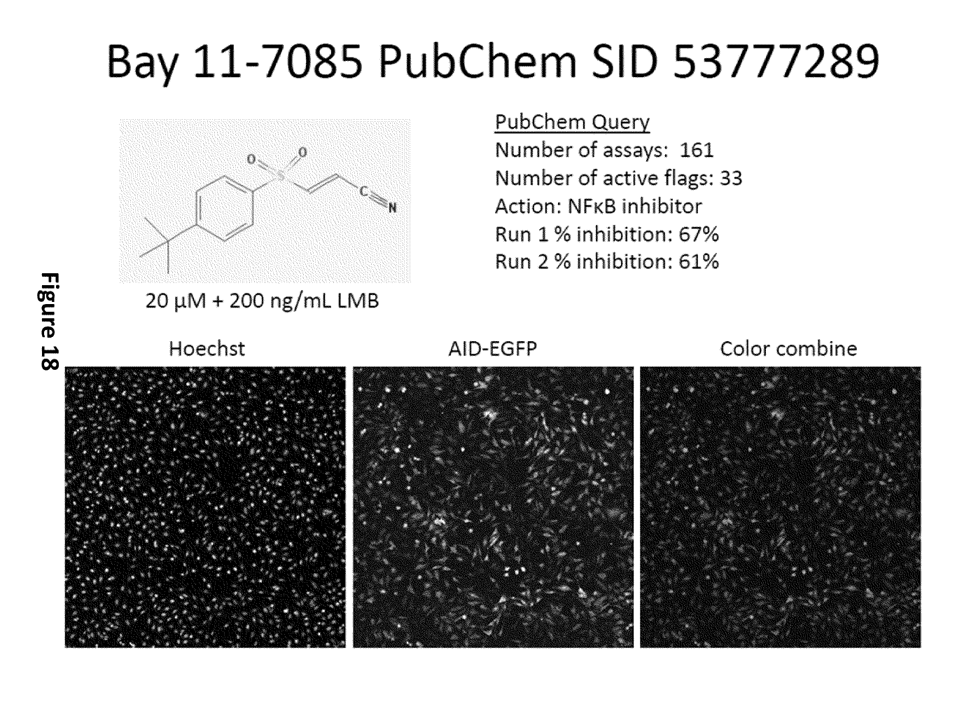

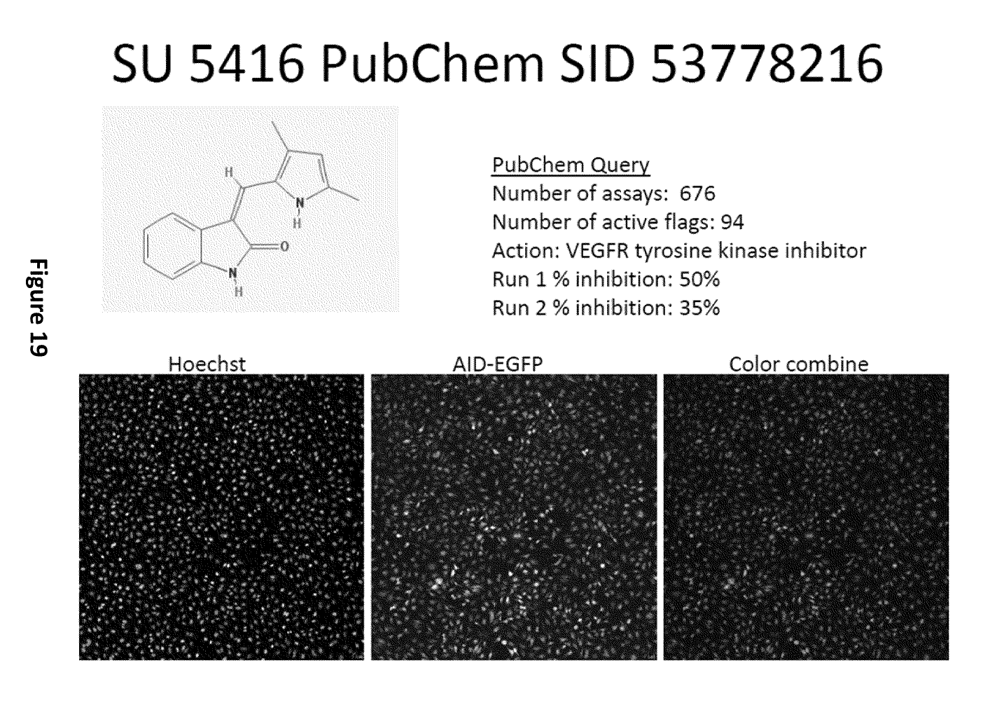

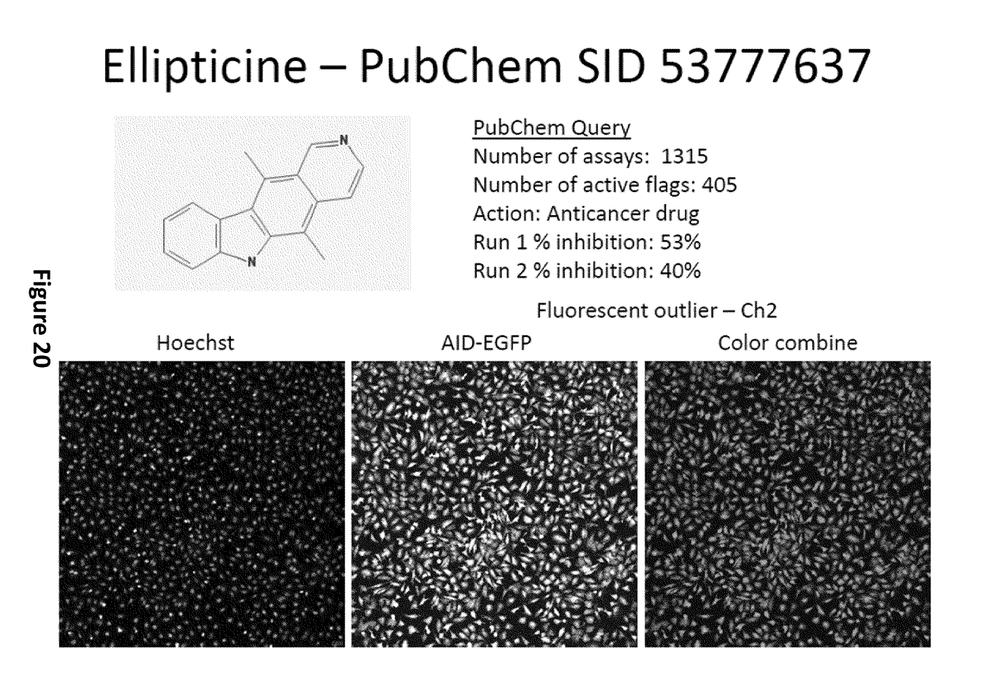

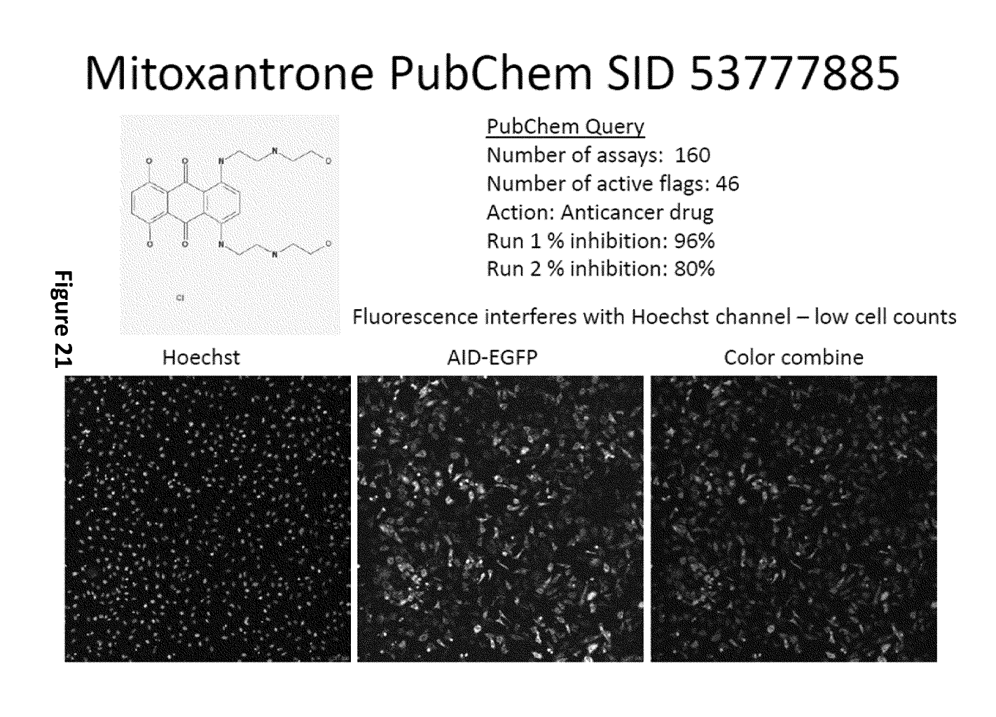

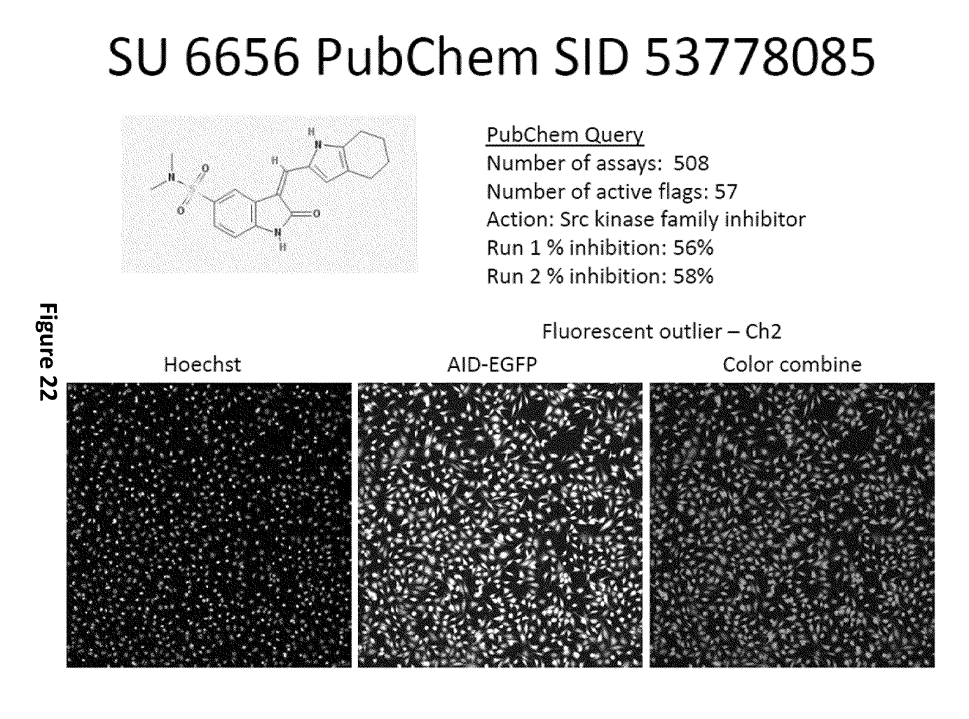

FIG. 18 through FIG. 22 depicts the structure of each identified compound, their mediated percent inhibition, and images of Hoechst and AID-EGFP (and color combine) from wells treated with the compound. Of note, Bay 11-7085 (Pubchem SID 53777289) is an NFκB inhibitor that was recently found to inhibit the nuclear import of the glucocorticoid receptor. Thus, while this compound may not be specific to AID, the identification of the compound validates that the present screen is robust to identify nuclear import inhibitors.

### Example 8

### Development of AID/TALDO1 Simultaneous Primary and Counter Screen

Life-cell assay with stable expression of fluorescent AID and TALDO1 as a Primary/Counter screen ultra-high throughput assay for identifying AID-selective nuclear import inhibitors is developed.

Ideally, compounds that selectively inhibit AID nuclear import will do so because they bind to AID in such a manner that they prevent interactions with the import machinery. Some AID nuclear import inhibitors may bind to importins α1, 3, and 5 or importin β and thereby inhibit AID nuclear import. Non-selective inhibition of the importin α/β chaperones might be cytotoxic because they will inhibit the nuclear import of many other proteins that rely on the same chaperones. Run in parallel with the other counter screens will be an in-cell assay wherein transaldolase 1 (TALDO1) a protein that uses the same importins as AID, is co-expressed with AID. In some instances, the use of TALDO1 as the internal or simultaneous counter screen is preferred as it uses the same transporters as AID. Thus, compounds that affect AID nuclear import and not TALDO1 have the highest probability of reacting with or binding to AID, rather than the transporter, nuclear pore complex, or anything else common to the import mechanism of TALDO1. Thus, compounds identified in an AID/TALDO1 primary/counter screen have the highest probability of being AID-specific nuclear import inhibitors, and therefore are least likely to have off-target and/or toxic effects. Compounds that inhibit the nuclear import of AID and not TALDO1 are prioritized.

A HeLa cell line stably expressing equivalent levels of AID-EGFP and mCherry-TALDO1 was established by transducing cells with a lentiviral vector encoding AID-EGFP. mCherry-TALDO1 was transfected/selected after the AID-EGFP line was sorted and selected. The cell line was then sorted for the co-expression of both proteins.

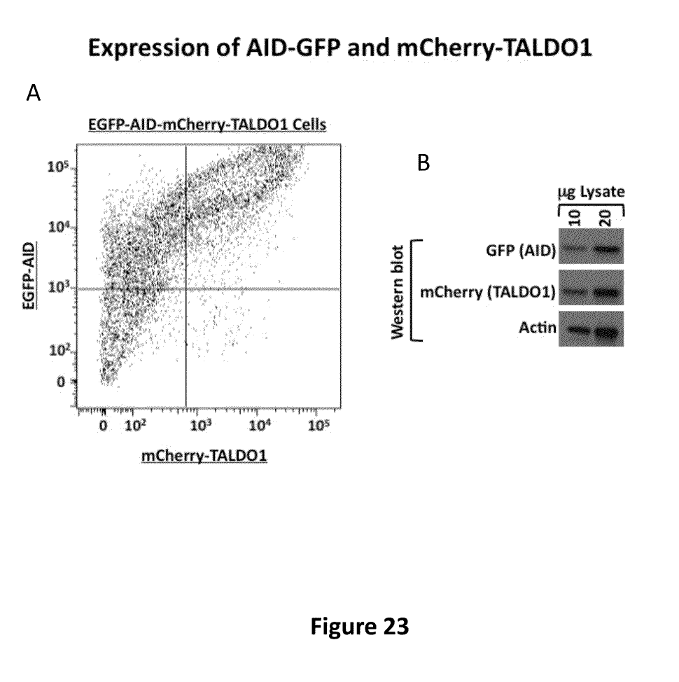

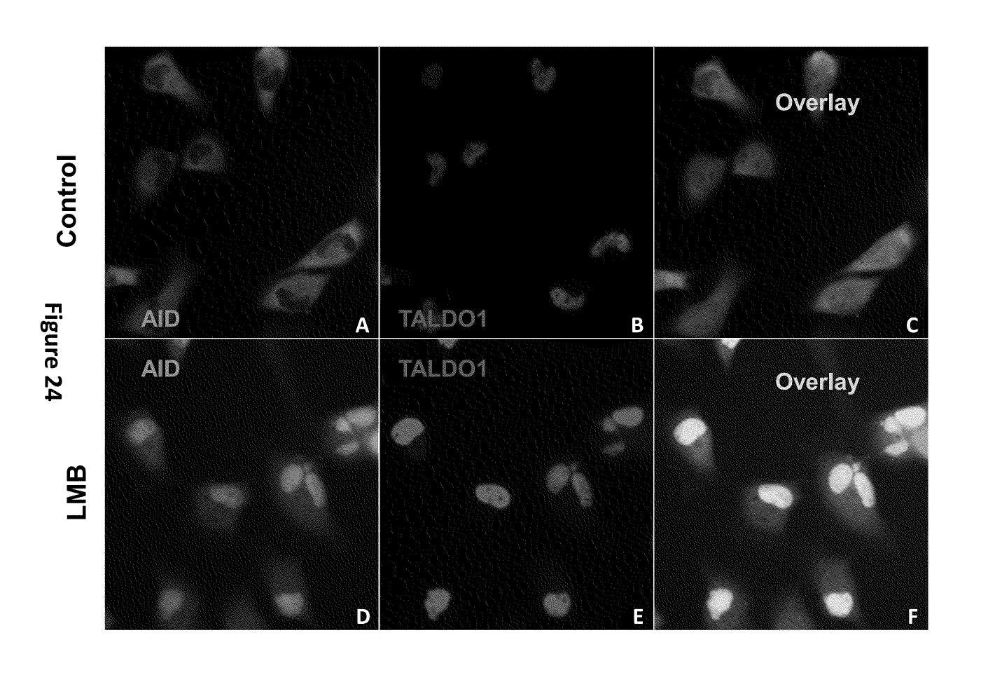

FIG. 23 depicts the expression of AID-EGFP and mCherry-TALDO1, as measured by flow cytometry (FIG. 23A) and western blot (FIG. 23B). The data presented therein, demonstrates that the produced cell line expresses both AID-EGFP and mCherry-TALDO1. FIG. 24 depicts the localization of AID-EGFP and mCherry-TALDO1 in control treated cells and LMB treated cells. While LMB treatment leads to AID-EGFP to shift localization from primarily cytoplasmic to primarily nuclear, mCherry-TALDO1 remained nuclear. This cell line is then used in a simultaneous primary/counter screen for compounds that selectively alter AID import, while not affecting TALDO1 localization.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of screening a library of compounds to provide a pool of specific and non-toxic nuclear import inhibitors for Activity-induced Deaminase (AID), said method comprising:
a) conducting a primary screen to evaluate an on-target effect of at least one compound from the library for the ability to inhibit nuclear import of AID, wherein the primary screen comprises administering the at least one compound from the library and a nuclear export inhibitor to a cell modified to comprise AID tagged with a detectable label;
b) conducting a counter screen to evaluate an off-target effect of the at least one compound from the library for the ability to alter the cellular localization of a non-AID protein, wherein the counter screen comprises administering the at least one compound from the library and the nuclear export inhibitor to a cell modified to comprise the non-AID protein tagged with a detectable label, wherein the non-AID protein is selected from the group consisting of histone H1 (H1), APOBEC1 Complementation Factor (ACF), heterogeneous nuclear ribonucleoprotein A1 (hnRNP A1), and transaldoase 1 (TALDO-1);
c) selecting a compound from the library that exhibits the ability to inhibit nuclear import of AID and does not substantially alter the cellular localization of the protein that is not AID, thereby providing a pool of specific and non-toxic nuclear import inhibitors for AID.

2. The method of claim 1, wherein the cell of the primary screen and the cell of the counter screen are the same cell, thereby allowing for a simultaneous primary screen and secondary screen.

3. The method of claim 1, wherein the nuclear export inhibitor is selected from the group consisting of LMB and Ratjadone A.

4. The method of claim 1, wherein evaluating an on-target effect in the primary screen comprises detecting the cellular localization of AID and comparing:
a) the cellular localization of AID detected in a control cell after administration of the nuclear export inhibitor and;
b) the cellular localization AID detected in the cell of the primary screen after the administration of both the nuclear export inhibitor and the compound,
wherein when the nuclear to cytoplasmic ratio (N/C) of AID in the control cell after administration of the nuclear export inhibitor is substantially greater than the N/C of AID in the cell of the primary screen after administration of the nuclear export inhibitor and the compound, the compound exhibits activity of inhibiting nuclear import of AID.

5. The method of claim 1, wherein evaluating an off-target effect in the counter screen comprises detecting the cellular localization of the non-AID protein and comparing:
a) the cellular localization of the non-AID protein in a control cell detected after administration of the nuclear export inhibitor and;
b) the cellular localization of the non-AID protein in the cell of the counter screen detected after the administration of both the nuclear export inhibitor and the compound,
wherein when the nuclear to cytoplasmic ratio (N/C) of the non-AID protein in the control cell after administration of the nuclear export inhibitor is substantially greater than the N/C of the non-AID protein in the cell of the counter screen after administration of the nuclear export inhibitor and the compound, the compound exhibits activity of altering the cellular localization of the non-AID protein.

6. The method of claim 1, further comprising conducting a counter screen to evaluate the toxicity of the at least one compound.

7. The method of claim 1, further comprising evaluating the ability for the at least one compound to inhibit somatic hypermutation (SHM)

8. The method of claim 1, further comprising evaluating the ability for the at least one compound to inhibit class switch recombination (CSR).

9. A compound selected from a pool of specific and non-toxic nuclear import inhibitors for AID, wherein the pool is identified by the method of claim 1, further wherein the compound inhibits the nuclear import of AID.

10. The compound of claim 9, wherein the compound prevents somatic hypermutation and class switch recombination.

11. The compound of claim 9, wherein the compound exhibits an anti-cancer property.

12. A method of treating a subject diagnosed with cancer or a subject at risk for developing cancer, said method comprising administering to the subject an effective amount of a compound that is an AID-selective inhibitor of nuclear import.

13. The method of claim 12, wherein the cancer is selected from the group consisting of Follicular Lymphoma, chronic lymphocytic leukemias, acute lymphoblastic leukemias, diffuse large B cell lymphomas, Burkiett's mantle cell lymphomas, Hodgkin's disease, and any combination thereof.

14. The method of claim 12, wherein the cancer is associated with a solid tumor of at least one member of the group consisting of the gastrointestinal system, urogenital system, breast, skin, and nervous system.

15. The method of claim 12, wherein the method inhibits the progression of cancer.

16. A method of screening a library to provide a pool of specific and non-toxic nuclear import inhibitors for a first protein wherein the first protein is capable of nuclear import, said method comprising:
a) conducting a primary screen to evaluate an on-target effect of at least one compound from the library for the ability to inhibit nuclear import of the first protein, wherein the primary screen comprises administering the at least one compound from the library and a nuclear export inhibitor to a cell comprising the first protein tagged with a detectable label;
b) conducting a counter screen to evaluate an off-target effect of the at least one compound from the library for the ability to altering the cellular localization of a second protein that is not the first protein, wherein the counter screen comprises administering the at least one compound from the library and a nuclear export inhibitor to a cell comprising a second protein tagged with a detectable label;
c) selecting a compound from the library that exhibits the ability to inhibit nuclear import of the first protein and does not substantially altering the cellular localization of the second protein, thereby providing a pool of specific and non-toxic nuclear import inhibitors for the first protein.

17. The method of claim 16, wherein the first protein is AID.

18. The method of claim 16, wherein the cell of the primary screen and the cell of the counter screen are the same cell, thereby allowing for a simultaneous primary screen and secondary screen.

19. The method of claim 16, wherein the nuclear export inhibitor is selected from the group consisting of LMB and Ratjadone A.

20. The method of claim 16, wherein evaluating an on-target effect in the primary screen comprises detecting the cellular localization of the first protein and comparing:
a) the cellular localization of the first protein in a control cell detected after administration of the nuclear export inhibitor and;
b) the cellular localization of the first protein in the cell of the primary screen detected after the administration of both the nuclear export inhibitor and the compound,
wherein when the nuclear to cytoplasmic ratio (N/C) of the first protein in the control cell after administration of the nuclear export inhibitor is substantially greater than the N/C of the first protein in the cell of the primary screen after administration of the nuclear export inhibitor and the compound, the compound exhibits activity of inhibiting nuclear import of the first protein.

21. The method of claim 16, wherein evaluating an off-target effect in the counter screen comprises detecting the cellular localization of the second protein and comparing:
a) the cellular localization of the second protein in a control cell detected after administration of the nuclear export inhibitor and;
b) the cellular localization of the second protein in the cell of the counter screen detected after the administration of both the nuclear export inhibitor and the compound,
wherein when the nuclear to cytoplasmic ratio (N/C) of the second protein in the control cell after administration of the nuclear export inhibitor is substantially greater than the N/C of the second protein in the cell of the second protein after administration of the nuclear export inhibitor and the compound, the compound exhibits activity of altering the cellular localization of the second protein.

22. The method of claim 16, wherein the second protein is selected from the group consisting of histone H1 (H1), APOBEC1 Complementation Factor (ACF), heterogeneous nuclear ribonucleoprotein A1 (hnRNP A1), and transaldoase 1 (TALDO-1).

23. The method of claim 16, wherein the primary screen and counter screen are conducted sequentially.

24. The method of claim 16, wherein the primary screen narrows the library to provide a second library comprising essentially of at least one compound that exhibits the activity of inhibiting nuclear import of the first protein, and wherein the counter screen evaluates off target effects of the at least one compound of the second library.

25. The method of claim 16, further comprising conducting a counter screen to evaluate the toxicity of the at least one compound.

26. The method of claim 16, further comprising evaluating the ability for the at least one compound to inhibit somatic hypermutation (SHM)

27. The method of claim 16, further comprising evaluating the ability for the at least one compound to inhibit class switch recombination (CSR).
